(19) United States
(12) Patent Application Publication
(10) Pub. No.: US 2006/0069115 A1
(43) Pub. Date: Mar. 30, 2006

(54) Use of pde4 inhibitors as adjunct therapy for psychiatric disorders

(76) Inventor: Edward M. Scolnick, Wynnewood, PA (US)

Correspondence Address: MERCK AND CO., INC, P O BOX 2000, RAHWAY, NJ, 07065-0907, US

(21) Appl. No.: 10/530,841

(22) PCT Filed: Nov. 10, 2003
(86) PCT No.: PCT/US03/35718

§ 371 Date: Apr. 8, 2005

Related U.S. Application Data
(60) Provisional application No. 60/426,529, filed on Nov. 15, 2002.

(51) Int. Cl. A61K 31/4745

(52) U.S. Cl. 514/300

(57) ABSTRACT

The use of a PDE4 inhibitor in conjunction with psychotherapy provides enhanced therapeutic results in the treatment of psychiatric disorders including, for example, specific phobias, panic disorders, anxiety disorders including posttraumatic stress disorders, and obsessive-compulsive disorder.

### BACKGROUND OF THE INVENTION

Classical fear conditioning occurs when an affectively neutral stimulus is paired with a noxious aversive stimulus (unconditioned stimulus (US)) such as footshock. Afterward, the previously neutral stimulus (i.e., now the conditioned stimulus (CS)) is able to elicit a variety of autonomic, hormonal, and skeletal responses that accompany the conscious experience of fear in humans and which are used to operationally define fear in laboratory animals. The fear-eliciting properties of the CS can be extinguished by repeatedly presenting the CS in the absence of the US. It is genereally believed that extinction does not reflect unlearning of the original association but involves instead the formation of new associations that compete with the previously conditioned response, i.e., extinction is not a process of forgetting but a form of active learning.

A reduced ability to extinguish intense fear memories is a significant clinical problem for a wide range of psychiatric disorders including specific phobias, panic disorder, and posttraumatic stress disorder. Because treatment of these disorders often relies upon the progressive extinction of fear memories, pharmacological enhancement of extinction could be of considerable clinical benefit in these conditions.

Hormones are compounds that variously affect cellular activity. In many respects, hormones act as messengers to trigger specific cellular responses and activities. Many effects produced by hormones, however, are not caused by the singular effect of just the hormone. Instead, the hormone first binds to a receptor, thereby triggering the release of a second compound that goes on to affect the cellular activity. In this scenario, the hormone is-known as the first messenger while the second compound is called the second messenger. Cyclic adenosine monophosphate (adenosine 3′,5′-cyclic monophosphate, “cAMP” or “cyclic AMP”) is known as a second messenger for hormones including epinephrine, glucagon, calcitonin, corticotrophin, lipotropin, luteinizing hormone, norepinephrine, parathyroid hormone, thyroid-stimulating hormone, and vasopressin. Thus, cAMP mediates cellular responses to hormones. Cyclic AMP also mediates cellular responses to various neurotransmitters.

Phosphodiesterases (“PDE”) are a family of enzymes that metabolize 3′,5′-cyclic nucleotides to 5′-nucleoside monophosphates, thereby terminating cAMP second messenger activity. A particular phosphodiesterase, phosphodiesterase-4 (“PDE4”, also known as “PDE-IV”), which is a high affinity, cAMP specific, type IV PDE, has generated interest as potential targets for the development of novel pharmaceuticals particularly as anti-asthmatic and anti-inflammatory compounds. In PCT International Application No. WO01/87281, certain PDE4 inhibitors are said to be useful in enhancing cognitive function.

Tully and Cavallieri disclose in U.S. Patent Application Publication No. WO02/0076398 a method of therapy for cognitive deficits associated with a central nervous system disorder and methods of enhancing cognitive performance by combining cognitive training protocols with administration of CREB pathway-enhancing agents. Tully and Cavallieri does not disclose the use of this combination therapy for psychiatric disorders

Davis et al disclose in PCT International Application No. WO02/078629 methods for treating an individual with a psychiatric disorder with a pharmacologic agent that enhances learning or conditioning in combination with a session of psychotherapy. Davis et al does not disclose the use of PDE4 inhibitors in combination with psychotherapy.

Reines et al disclose in PCT International Application No. WO01/64223 the combination of a neurokinin-1 antagonist or an alpha-2 adrenoreceptor agonist with a PDE4 inhibitor for the treatment or prevention of depression and/or anxiety. Reines et al does not disclose the use of PDE4 inhibitors to augment the effect of psychotherapy.

### SUMMARY OF THE INVENTION

The present invention provides a method for the treatment of psychiatric disorders using a PDE4 inhibitor in conjunction with psychotherapy. The inclusion of a PDE4 inhibitor in the treatment modality enhances the effectiveness of psychotherapy resulting in fewer sessions required to achieve improvement, shorter intervals between sessions, or more pronounced improvement of symptoms, as compared to psychotherapy alone.

### DETAILED DESCRIPTION OF THE INVENTION

The present invention provides a method for the treatment of psychiatric disorder in a patient, said method comprises administering to said patient a therapeutically effective amount of a PDE4 inhibitor in combination with psychotherapy. The method comprises subjecting the individual to one or more sessions of a combination therapy protocol, where the combination therapy protocol comprises administering a therapeutically effective amount of a PDE4 inhibitor in combination with a session of psychotherapy.

The term “psychiatric disorder,” as used herein, refers to a disorder that can be treated with the methods of the present invention. For purposes of the present invention, an individual said to have a psychiatric disorder will have one or more disorders that can be treated with the methods of the present invention. Thus an individiual may have a single disorder, or may have a constellation of disorders that are to be treated by the methods described herein. The psychiatric disorders contemplated in the present invention include, but are not limited to, fear and anxiety disorders, addictive disorders including substance abuse disorders, and mood disorders. Within the fear and anxiety disorder category, the invention encompasses the treatment of panic disorder, specific phobia, posttraumatic stress disorder (PTSD), obsessive-compulsive disorder, and movement disorder such as Tourette's syndrome. The disorders contemplated herein are defined in, for example, the DSM-IV (Diagnostic and Statistical Manual; 4th edition, American Psychiatric Association).

In one aspect of the present invention, the psychiatric disorder to be treated is PTSD. Posttraumatic stress disorder (PTSD) is defined by DSM-IV as an anxiety disorder that an individual may develop following exposure to a traumatic event, and is characterized by (1) reexperiencing the traumatic event, such as recurrent nightmares, intrusive recollections of the event, flashbacks, physiological and psychological responses to internal or external cues relating to the event, etc; (2) persistent avoidance of thoughts, people or places associated with the event; (3) numbing of general responsiveness such as emotional detachment, restricted affect or loss of interest in activities; and (4) persistence of increased arousal such as exaggerated startle response, hypervigilence, irritability, difficulty sleeping, etc. In the US the lifetime prevalence of PTSD is at least 1%, and in high-risk populations, such as combat veterans or victims of criminal violence, prevalence is reported to be between 3 and 58%; PTSD is therefore of considerable public health concern.

The methods of the invention encompass the use of any type of psychotherapy that is suitable for the particular psychiatric disorder for which the individual is undergoing treatment, and may be conducted in one or more sessions. Suitable methods of psychotherapy include behavior psychotherapy such as exposure-based psychotherapy, cognitive psychotherapy including cognitive training and psychodynamically oriented psychotherapy (see, for example, Foa (2000) J. Clin. Psych. 61(suppl. 5):43-38). Exposure based psychotherapy include for example, systematic desensitization, flooding, implosive therapy, and extinction-based therapy. Such psychotherapy modalities are well known to one skilled in the art of psychiatry.

One method of psychotherapy specifically contemplated is the use of virtual reality (VR) exposure therapy to treat a psychiatric disorder using the combination therapy protocol of the invention. VR exposure therapy has been used to treat a variety of disorders including anxiety disorders such as the fear of heights (Rothbaum and Hodges (1999) Behav. Modif 23(4):507-25), as well as specific phobias, eating disorders, and PTSD (Anderson et al. (2001) Bull. Menninger Clin. 65(1):78-91). Because of the prevalence of PTSD in the general population and the successful use of VR therapy to treat PTSD in, for example, Vietnam veterans (Rothbaum et al. 30 (1999) J. Trauma Stress 12(2):263-71) or rape victims (Rothbaum et al. (2001) J. Trauma Stress 14(2):283-93), one embodiment of the present invention specifically contemplates the use of such VR exposure psychotherapy in combination with a PDE4 inhibitor as described elsewhere herein to treat PTSD.

The PDE4 inhibitors used to practice the present invention may be any that is known, or discovered to inhibit the PDE4 enzyme, and are not limited to any particular structural class of compounds. As used herein, the term “PDE4 inhibitors” includes any pharmaceutically acceptable salts thereof. The assay for identifying PDE4 inhibitors is described in the Examples section hereinbelow. The utility of PDE4 inhibitors in the present invention may be evaluated using the animal fear conditioning/extinction and clinical experimental protocols disclosed in PCT Application No. WO02/078629, which is hereby incorporated by reference, with the exception that a PDE4 inhibitor is used instead of the pharmacological agent used therein.

The PDE4 inhibitor may be peptidal or non-peptidal in nature; however, the use of a non-peptidal PDE4 inhibitor is preferred. In a preferred embodiment, the PDE4 inhibitor is a CNS-penetrant PDE4 inhibitor. In addition, for convenience the use of an orally active PDE4 inhibitor is preferred. To facilitate dosing, it is also preferred that the PDE4 inhibitor is a long acting PDE4 inhibitor. An especially preferred class of PDE4 inhibitors of use in the present invention are those compounds which are orally active and long acting. Representative PDE4 inhibitors of use in the present invention are fully described, for example, in U.S. Pat. Nos. 5,340,827, 5,550,137, 5,491,147, 5,608,070, 5,622,977, 5,633,257, 5,712,298, 5,739,144, 5,776,958, 5,780,477, 5,780,478, 5,786,354, 5,798,373, 5,580,888, 5,849,770, 5,859,034, 5,866,593, 5,891,896, 5,919,801, 6,005,118, 6,410,563, 6,399,639, 6,448,274 and International Patent Publications WO 94/22852, WO 95/35283, WO 96/00215. Suitable PDE4 inhibitors include pentoxifylline, isobutylmethylxanthine, cilomilast, roflumilast and its N-oxide, arofylline, lirimilast, GW84247, CP-671305, and terferol. Other suitable PDE4 inhibitors for use in the present invention are those presented in the Examples section and their pharmaceutically acceptable salts.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are benzenesulfonic, citric, hydrobromic, hydrochloric, maleic, phosphoric, sulfuric, and tartaric acids.

The PDE4 inhibitor may be administered to the patient prior to, during or after the psychotherapy session. It is preferably administered within about 24 hours prior to or following the session of psychotherapy, more preferably within about 24 hour prior to initiating psychotherapy, and even more preferably within about 12 hours prior to initiating psychotherapy. A full course of treatment of psychiatric disorder entails at least one session of this combination therapy protocol.

The PDE4 inhibitor may be administered in a composition suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The PDE4 inhibitor is administered in a therapeutically effective amount, which is that amount that provides improvied therapeutic benefit relative to that achieved by psychotherapy alone. Dosage levels from about 0.001 mg/kg to about 140 mg/kg of body weight per day are useful for the purpose of the present invention or about 0.05 mg to about 7 g per patient per day. Alternatively, dosage levels from about about 0.01 mg to 50 mg of the compound per kilogram of body weight per day, or alternatively about 0.5 mg to about 2.5 g per patient per day.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 0.01 mg to about 1000 mg of the active ingredient, typically 0.01 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In practice, the PDE4 inhibitors can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the PDE4 inhibitor may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of a compound of the Examples. The compounds or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices prepared via conventional processes As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a PDE4 inhibitor may also be prepared in powder or liquid concentrate form.

Further, the compound of this invention can be utilized in combination with other therapeutic compounds. In particular, the combinations of the PDE4 inhibiting compound of this invention can be advantageously used in combination with i) Leukotriene receptor antagonists, ii) Leukotriene biosynthesis inhibitors, iii) COX-2 selective inhibitors, iv) statins, v) NSAIDs, vi) M2/M3 antagonists, vii) corticosteroids, viii) H1 (histamine) receptor antagonists and ix) beta 2 adrenoceptor agonist.

Thus, for example, the PDE4 inhibitor may be administered with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered prior to, during or after a session of psychotherapy.

A subject undergoing treatment with the methods of the invention exhibits an improvement in one or more symptoms associated with the psychiatric disorder. For a description of the relevant symptoms, see, for example, the DSM-IV ((1994) Diagnostic and Statistical Mamlal of Mental Disorders (4th ea., American Psychiatric Association, Washington D.C.)), which is herein incorporated by reference. The efficacy of the methods of the invention can be assessed using any clinically recognized assessment method for measuring a reduction of one or more symptoms of the particular psychiatric disorder. Examples of such assessment methods are described in, for example, in Experiment 7 of PCT Application WO02/078629.

The present invention may be better understood with reference to the following examples. These examples are intended to be representative of specific embodiments of the invention, and are not intended as limiting the scope of the invention.

### EXAMPLES

### SPA (Scintillation Proximity Assay) Based PDE Activity Assay Protocol

Compounds which inhibit the hydrolysis of cAMP to AMP by the type-IV cAMP-specific phosphodiesterases may be screened in a 96-well plate format as follows:

In a 96 well-plate at 30° C. is added the test compound (dissolved in 2 μL DMSO), 188 mL of substrate buffer containing [2,8-³H] adenosine 3′,5′-cyclic phosphate (cAMP, 100 nM to 50 μM), 10 mM MgCl₂, 1 mM EDTA, 50 mM Tris, pH 7.5. The reaction is initiated by the addition of 10 mL of human recombinant PDE4 (the amount was controlled so that ˜10% product was formed in 10 min.). The reaction is stopped after 10 min. by the addition of 1 mg of PDE-SPA beads (Amersham Pharmacia Biotech, Inc., Piscataway, N.J.). The product AMP generated is quantified on a Wallac Microbeta® 96-well plate counter (EG&G Wallac Co., Gaithersburg, Md.). The signal in the absence of enzyme is defined as the background. 100% activity is defined as the signal detected in the presence of enzyme and DMSO with the background subtracted. Percentage of inhibition is calculated accordingly. IC₅₀ value is approximated with a non-linear regression fit using the standard 4-parameter/multiple binding sites equation from a ten point titration.

### COMPOUND EXAMPLES

The compound examples are comprised of four sub-sets—Example set A, Example set B, and Example set C.

The compounds of Examples 1A through 42A are characterized and prepared as disclosed in U.S. Pat. No. 6,410,563 B1, issued Jun. 25, 2002, which is hereby iincorporated by reference.

1A. and 2A. 6-isopropyl-8-(3-{(Z/E)-2-[4-(methylsulfonyl)phenyl]-2-phenylethenyl}phenyl)quinoline;

3A. 6-isopropyl-8-{3-[(E/Z)-2-[4-(methylsulfonyl)phenyl]-2-(1,3-thiazol-2-yl)ethenyl]phenyl}quinoline;

4A. 6-isopropyl-8-(3-{(E)-2-(1-methyl-1H-imidazol-2-yl)-2-[4-(methylsulfonyl)phenyl]ethenyl}phenyl)quinoline;

5A. and 6A. 6-isopropyl-8-(3-{(Z/E)-2-(4-fluorophenyl)-2-[4-(methylsulfonyl)phenyl]ethenyl}phenyl)quinoline;

7A. 2-(2-{(E/Z)-2-[3-(6-isopropyl-8-quinolinyl)phenyl]-1-[4-(methylsulfonyl)phenyl]ethenyl}-1,3-thiazol-5-yl)-2-propanol;

8A. 2-[8-(3-{(E/Z)-2-[5-(1-hydroxy-1-methylethyl)-1,3-thiazol-2-yl]-2-[4-(methylsulfonyl)phenyl]ethenyl}phenyl)-6-quinolinyl]-2-methylpropanenitrile;

9A. 2-methyl-2-[8-(3-{(E)-2-(1-methyl-1H-imidazol-2-yl)-2-[4-(methylsulfonyl)phenyl]ethenyl}phenyl)-6-quinolinyl]propanenitrile;

10A. 6-[1-(methylsulfonyl)ethyl]-8-{3-[(E)-2-[4-(methylsulfonyl)phenyl]-2-(1,3-thiazol-2-yl)ethenyl]phenyl}quinoline;

11A. 6-[1-methyl-1-(methylsulfonyl)ethyl]-8-{3-[(E)-2-[4-(methylsulfonyl)phenyl]-2-(1,3-thiazol-2-yl)ethenyl]phenyl}quinoline;

12A. 8-(3-{(Z)-2-(1-methyl-1H-imidazol-2-yl)-2-[4-(methylsulfonyl)phenyl]ethenyl}phenyl)-6-[1-(methylsulfonyl)ethyl]quinoline;

13A. 8-(3-{(Z)-2-(1-methyl-1H-imidazol-2-yl)-2-[4-(methylsulfonyl)-phenyl]ethenyl}phenyl)-6-[1-methyl-1-(methylsulfonyl)ethyl]quinoline;

14A. and 15. 6-[1-methyl-1-(methylsulfonyl)ethyl]-8-(3-{(E/Z)-2-(3-methyl-1,2,4-oxadiazol-5-yl)-2-[4-(methylsulfonyl)phenyl]ethenyl}phenyl)quinoline;

16A. and 17A. (E/Z)-3-{3-[6-(1-cyano-1-methylethyl)-8-quinolinyl]phenyl}-N-isopropyl-2-[4-(methylsulfonyl)phenyl]-2-propenamide;

18A. 8-(3-{(E)-2-{3-[(4-methoxyphenoxy)methyl]-1,2,4-oxadiazol-5-yl}-2-[4-(methylsulfonyl)phenyl]ethenyl}phenyl)-6-[1-methyl-1-(methylsulfonyl)ethyl]quinoline;

19A. (5-{(E)-2-(3-{6-[1-methyl-1-(methylsulfonyl)ethyl]-8-quinolinyl}phenyl)-1-[4-(methylsulfonyl)phenyl]ethenyl}-1,2,4-oxadiazol-3-yl)methanol;

20A. (E)-N-isopropyl-3-(3-{6-[1-methyl-1-(methylsulfonyl)ethyl]-8-quinolinyl}phenyl)-2-[4-(methylsulfonyl)phenyl]-2-propenamide;

21A. (E)-3-{3-[6-(1-cyano-1-methylethyl)-8-quinolinyl]phenyl}-2-[4-(methylsulfonyl)phenyl]-2-propenoic acid;

22A. 2-methyl-2-[8-(3-{(E)-2-(3-methyl-1,2,4-oxadiazol-5-yl)-2-[4-(methylsulfonyl)phenyl]ethenyl}phenyl)-6-quinolinyl]propanenitrile;

23A. (E)-3-{3-[6-(1-cyano-1-methylethyl)-8-quinolinyl]phenyl}-2-[4-(methylsulfonyl)phenyl]-2-propenamide;

24A. (E)-N-(tert-butyl)-3-{3-[6-(1-cyano-1-methylethyl)-8-quinolinyl]phenyl}-2-[4-(methylsulfonyl)phenyl]-2-propenamide;

25A. (E)-3-[3-(6-isopropyl-8-quinolinyl)phenyl]-2-[4-(methylsulfonyl)phenyl]-2-propenoic acid;

26A. 6-isopropyl-8-(3-{(E)-2-(3-methyl-1,2,4-oxadiazol-5-yl)-2-[4-(methylsulfonyl)phenyl]ethenyl}phenyl)quinoline;

27A. (E)-3-(3-{6-[1-methyl-1-(methylsulfonyl)ethyl]-8-quinolinyl}phenyl)-2-[4-(methylsulfonyl)phenyl]-1-(1-pyrrolidinyl)-2-propen-1-one;

28A. (E)-N-cyclopropyl-3-(3-{6-[1-methyl-1-(methylsulfonyl)ethyl]-8-quinolinyl}phenyl)-2-[4-(methylsulfonyl)phenyl]-2-propenamide;

29A. (E)-N-(tert-butyl)-3-(3-{6-[1-methyl-1-(methylsulfonyl)ethyl]-8-quinolinyl}phenyl)-2-[4-(methylsulfonyl)phenyl]-2-propenamide;

30A. 8-{3-[2,2-bis(4-chlorophenyl)vinyl]phenyl}-6-isopropylquinoline;

31A. and 32A. 6-isopropyl-8-(3-{(E/Z)-2-(6-methyl-3-pyridinyl)-2-[4-(methylsulfonyl)phenyl]ethenyl}phenyl)quinoline;

33A. and 34A. 6-isopropyl-8-(3-{(E/Z)-2-(5-methyl-2-pyridinyl)-2-[4-(methylsulfonyl)phenyl]ethenyl}phenyl)quinoline;

35A. 8-(3-{2,2-bis[4-(methylsulfonyl)phenyl]vinyl}phenyl)-6-isopropylquinoline;

36A. and 37A. 2-methyl-2-[8-(3-{(E/Z)-2-(5-methyl-2-pyridinyl)-2-[4-(methylsulfonyl)phenyl]ethenyl}phenyl)-6-quinolinyl]propanenitrile;

38A. 2-[8-(3-{2,2-bis[4-(methylsulfonyl)phenyl]vinyl}phenyl)-6-quinolinyl]-2-methylpropanenitrile;

39A. 2-methyl-2-(8-{3-[(E)-2-[4-(methylsulfonyl)phenyl]-2-(2-pyridinyl)ethenyl]phenyl}-6-quinolinyl)propanenitrile;

40A. and 41A. 6-[1-methyl-1-(methylsulfonyl)ethyl]-8-(3-{(E/Z)-2-(5-methyl-2-pyridinyl)-2-[4-(methylsulfonyl)phenyl]ethenyl}phenyl)quinoline;

42A. 2-(6-{(E)-2-(3-{6-[1-methyl-1-(methylsulfonyl)ethyl)-8-quinolinyl}phenyl)-1-[4(methylsulfonyl)phenyl]ethenyl}-3-pyridinyl)-2-propanol.

The compounds of Examples 1B through 32b are characterized and prepared as disclosed in U.S. Pat. No. 6,399,636 B2, issued Jun. 4, 2002, which is hereby incorporated by reference.
1B (±)-4-{2-[3,4-Bis(difluoromethoxy)phenyl]-2-{5-[2-(1-hydroxy-1-methyl)-ethyl]thiazolyl}ethyl}pyridine N-oxide;
2B chiral 4-{2-[3,4-Bis(difluoromethoxy)phenyl]-2-{5-[2-(1-hydroxy-1-methyl)ethyl]thiazolyl}ethyl}pyridine N-oxide;
3B (±/±)-4-{2-[3,4-Bis(difluoromethoxy)phenyl]-2-[5-(2-(1-hydroxy-2,2,2-trifluoro)ethyl)thiazolyl]ethyl}pyridine;
4B (±/±)-4-{2-[3,4-Bis(difluoromethoxy)phenyl]-2-[5-(2-(1-hydroxy-2,2,2-trifluoro)ethyl)thiazolyl]ethyl}pyridine N-oxide;
5B (±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine;
6B (±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine N-oxide;
7B (±/±)-4-{2-[3,4-Bis(difluoromethoxy)phenyl]-2-{5-[2-(1-hydroxy-1-trifluoromethyl)ethyl]thiazolyl}ethyl}pyridine N-oxide;
8B (±/±)-4-{2-[3,4-Bis(difluoromethoxy)phenyl]-2-[5-(2-phenylmethanol)-thiazolyl]ethyl}pyridine N-oxide;
9B (±/±)-4-{2-[3,4-Bis(difluoromethoxy)phenyl]-2-[5-(2-(1-hydroxy-1-phenyl)ethyl)thiazolyl]ethyl}pyridine N-oxide;
10B (±/±)-4-{2-[3,4-Bis(difluoromethoxy)phenyl]-2-[5-(2-(1-hydroxy-1-phenyl-2,2,2-trifluoro)ethyl)thiazolyl]ethyl}pyridine N-oxide;
11B (±/±)-4-{2-[3,4-Bis(difluoromethoxy)phenyl]-2-[5-(2-(1-hydroxy-1-phenyl)propyl)thiazolyl]ethyl}pyridine N-oxide;
12B (±/±)-4-{2-[3,4-Bis(difluoromethoxy)phenyl]-2-[5-(2-cyclohexylmethanol)-thiazolyl]ethyl}pyridine;
13B (±/±)-4-{2-[3,4-Bis(difluoromethoxy)phenyl]-2-[5-(2-(1-hydroxy-1-cyclohexyl-2,2,2-trifluoromethyl)ethyl)thiazolyl]ethyl}pyridine N-oxide;
14B (±/±)-4-{2-[3,4-Bis(difluoromethoxy)phenyl]-2-[5-(2-(1-hydroxy-1-(4-ethyl)phenyl)ethyl)thiazolyl]ethyl}pyridine N-oxide;
15B (±/±)-4-{2-[3,4-Bis(difluoromethoxy)phenyl]-2-[5-(2-(1-hydroxy-1-(4-ethyl)phenyl-2,2,2-trifluoro)ethyl)thiazolyl]ethyl}pyridine N-oxide;
16B (±/±)-4-{2-[3,4-Bis(difluoromethoxy)phenyl]-2-[5-(2-(1-hydroxy-1-(4-fluoro)phenyl)ethyl)thiazolyl]ethyl}pyridine N-oxide;
17B (±/±)-4-{2-[3,4-Bis(difluoromethoxy)phenyl]-2-[5-(2-(1-hydroxy-1-(4-fluoro)phenyl-2,2,2-trifluoro)ethyl)thiazolyl]ethyl}pyridine N-oxide;
18B (±/±)-4-{2-[3,4-Bis(difluoromethoxy)phenyl]-2-[5-(2-(1-hydroxy-1-(5-bromopyridin-2-yl)-2,2,2-trifluoro)ethyl)thiazolyl]ethyl}pyridine N-oxide;
19B (±/±)-4-{2-[3,4-Bis(difluoromethoxy)phenyl]-2-[5-(2-(1-hydroxy-1-(6-bromopyridin-3-yl)-2,2,2-trifluoro)ethyl)thiazolyl]ethyl}pyridine N-oxide;
20B (±)-4-{2-[3,4-Bis(difluoromethoxy)phenyl]-2-{5-[2-(1-hydroxy)cyclobutyl]-thiazolyl}ethyl}pyridine N-oxide;
21B (±)-4-{2-[3,4-Bis(difluoromethoxy)phenyl]-2-{5-[2-(1-hydroxy)cyclohexyl]-thiazolyl}ethyl}pyridine N-oxide;
22B (±)-4-{2-[(3-cyclobutyloxy-4-difluoromethoxy)phenyl]-2-{5-[2-(1-hydroxy-1-methyl)ethyl]thiazolyl}ethyl}pyridine N-oxide;
23B chiral 4-{2-[(3-cyclobutyloxy-4-difluoromethoxy)phenyl]-2-{5-[2-(1-hydroxy-1-methyl)ethyl]thiazolyl}ethyl}pyridine N-oxide;
24B (±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine;
25B (±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
26B Chiral 3-{2-[(3-cyclobutyloxy-4-difluoromethoxy)phenyl]-2-{5-[2-(1-hydroxy-1-methyl)ethyl]thiazolyl}ethyl}pyridine;
27B Chiral 3-{2-[(3-cyclobutyloxy-4-difluoromethoxy)phenyl]-2-{5-[2-(1-hydroxy-1-methyl)ethyl]thiazolyl}ethyl}pyridine N-oxide;
28B Chiral 3-{2-[(3-cyclobutyloxy-4-difluoromethoxy)phenyl]-2-{5-[2-(1-hydroxy-1-methyl)ethyl]thiazolyl}ethyl}pyridine N-oxide;
29B Chiral 3-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
30B (±)-4-{2-[(3-cyclopropyloxy-4-difluoromethoxy)phenyl]-2-{5-[2-(1-hydroxy-1-methyl)ethyl]thiazolyl}ethyl}pyridine N-oxide;
31B (±)-3-{2-[(3-cyclopropyloxy-4-difluoromethoxy)phenyl]-2-{5-[2-(1-hydroxy-1-trifluoro methyl-2,2,2-trifluoro)ethyl]thiazolyl}ethyl}pyridine N-oxide; and
32B chiral 3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide.
   The compounds of Example 1C to 34C characterized and prepared as disclosed in WO03/18579A1

### EXAMPLE 1C

### N-Isopropyl-1-[3-(phenylethynyl)phenyl]-1,4-dihydro[1,8]naphthyridin-4-one-3-carboxamide

Step 1: Ethyl 3-(3-bromoanilino)-2-(2-chloronicotinoyl) acrylate.

A mixture of ethyl 2-chloronicotinoyl acetate (41.1 g, 180.5 mmol), triethyl orthoformate (40.12 g, 271 mmol) and acetic anhydride (92.05 g, 902.5 mmol) was heated at 130° C. for 2.5 hours. The volatile components were distilled off and the resulting residue was co-evaporated twice with xylene. The oily residue was dissolved in methylene chloride (250 mL) and 3-bromoaniline (37.25 g, 216.6 mmol) was added slowly. The resulting solution was stirred at room temperature for 18 hours, and the solvent evaporated away. The resulting crude compound was used as such in the next step.

Step 2: Ethyl 1-(3-bromophenyl)-1,4-dihydro[1,8]naphthyridin-4-one-3-carboxylate.

The crude compound from Step 1 was dissolved in tetrahydrofuran (500 mL), the solution was cooled to 0° C., and sodium hydride (as a 60% dispersion in oil , 9.4 g, 235 mmol) was added in portions. After stirring at 0° for 1 hour, the resulting mixture was allowed to warm up to room temperature. After 2 hours, water (400 mL) was added to the resulting suspension and the insoluble solid was filtered and washed copiously with water. When dry, the solid was stirred in ether (150 mL) at room temperature for 24 hours and filtered to afford the ethyl 1-(3-bromophenyl)-1,4-dihydro[1,8]naphthyridin-4-one-3-carboxylate compound as a cream-colored solid. 1H NMR (Acetone-d₆) δ 1.32 (t, 3H), 4.29 (q, 2H), 7.54-7.63 (m, 2H), 7.69 (dd, 1H), 7.78 (dd, 1H), 7.93 (s, 1H), 8.66-8.71 (m, 3H).

Step 3: 1-(3-Bromophenyl)-1,4-dihydro[1,8]naphthyridin-4-one-3-carboxylic acid.

A suspension of ethyl 1-(3-bromophenyl)-1,4-dihydro[1,8]naphthyridin-4-one-3-carboxylate from Step 2 (52.5 g, 140.7 mmol) in a mixture of tetrahydrofuran (400 mL), methanol (400 mL) and 1N aqueous sodium hydroxide (280 mL) was heated at ca 50° C. with stirring for 20 minutes. After cooling, the mixture was diluted with water (300 mL) and 1N aqueous HCl (325 mL) was added. After stirring for 45 minutes, the precipitate was filtered, washed well with water and dried to afford the 1-(3-bromophenyl)-1,4-dihydro[1,8]naphthyridin-4-one-3-carboxylic acid as a cream-colored solid. 1H NMR (Acetone-d₆) δ 7.65 (t, 1 H), 7.76 (m, 2H), 7.84 (d, 1H), 7.99 (s, 1H), 8.87 (m, 2H), 9.01 (s, 1H).

Step 4: N-Isopropyl-1-(3-bromophenyl)-1,4-dihydro[1,8]naphthyridin-4-one-3-carboxamide.

To a suspension of 1-(3-bromophenyl)-1,4-dihydro[1,8]naphthyridin-4-one-3-carboxylic acid from Step 3 (26.3 g, 76 mmol) and triethylamine (23.2 g, 230 mmol) in tetrahydrofuran (1000 mL) at 0° C. was added isobutyl chloroformate (18.85 g, 138 mmol). After stirring at 0° C. for 2 hours, isopropylamine (23 g, 390 mmol) was added and the mixture was allowed to warm up to room temperature and stirred overnight. The mixture was then partitioned between ethyl acetate and water, the organic phase was dried and evaporated to a solid which was stirred in ether at room temperature for 3 hours and filtered to afford the N-isopropyl-1-(3-bromophenyl)-1,4-dihydro[1,8]naphthyridin-4-one-3-carboxamide as a white solid. 1H NMR (Acetone-d₆) δ 1.25 (d, 6H), 4.17 (m, 1H), 7.59-7.63 (m, 2H), 7.70 (d, 1H), 7.80 (d, 1H), 7.94 (s, 1H), 8.73 (m, 1H), 8.78 (d, 1H), 8.85 (s, 1H), 9.61 (br, NH).

Step 5: N-Isopropyl-1-(3-phenylethynyl)phenyl]-1,4-dihydro[1,8]naphthyridin-4-one-3-carboxamide.

A mixture of amide from Step 4, phenylacetylene (1.9 eq), triethylamine (1.6 eq), triphenylphosphine (0.06 eq) and bis(triphenylphosphine)palladium (II) chloride (0.05 eq) in THF (16 mL/mmol) was stirred at room temperature for 20 minutes. Copper (I) iodide (5 mg/mmol) was added and the mixture was stirred at reflux for 18 hours. After cooling, the mixture was quenched with saturated aqueous ammonium chloride solution and partitioned between ethyl acetate and water. The organic phase was dried over magnesium sulfate and the crude product was chromatographed on silica gel eluting with a 1:9 mixture of ether and methylene chloride to afford a solid which was stirred in ether at room temperature and filtered to yield the N-Isopropyl-1-[(3-phenylethynyl)phenyl]-1,4-dihydro[1,8]naphthyridin-4-one-3-carboxamide compound as a solid. 1H NMR (Acetone-d₆) δ 1.24 (d, 6H), 4.18 (m, 1H), 7.42 (m, 3H), 7.56-7.61 (m, 3H), 7.69 (m, 2H), 7.76 (m, 1H), 7.85 (s, 1H), 8.73 (m, 1H), 8.77 (dd, 1H), 8.88 (s, 1H), 9.62 (br, NH).

### EXAMPLE 2C

### N-Isopropyl-1-[3-(2-pyridinylethynyl)phenyl]-1,4-dihydro[1,8]naphthyridin-4-one-3-carboxamide

Following the procedure of Step 5 of EXAMPLE 1C, but substituting 2-ethynylpyridine for phenylacetylene, the title compound was obtained as a brown solid. 1H NMR (Acetone-d₆) δ 1.25 (d, 6H), 4.18 (m, 1H), 7.38 (m, 1H), 7.59-7.64 (m, 2H), 7.71-7.76 (m, 2H), 7.81-7.85 (m, 2H), 7.92 (s, 1H), 8.61 (m, 1H), 8.74 (m, 1H), 8.78 (dd, 1H), 8.89 (s, 1H), 9.62 (br, NH).

### EXAMPLE 3C

### N-Isopropyl-1-[3-(4-pyridinylethynyl)phenyl]-1,4-dihydro[1,8]naphthyridin-4-one-3-carboxamide

Following the procedure of Step 5 of EXAMPLE 1C, but substituting 4-ethynylpyridine (J. Org. Chem. 1996, 61, 6535) for phenylacetylene, the title compound was obtained as a solid. 1H NMR (Acetone-d₆) δ 1.24 (d, 6H), 4.18 (m, 1H), 7.49 (m, 2H), 7.61 (m, 1H), 7.71-7.78 (m, 2H), 7.81 (m, 1H), 7.92 (s, 1H), 8.62 (m, 2H), 8.73 (m, 1H), 8.78 (dd, 1H), 8.87 (s, 1H), 9.62 (br, NH).

### EXAMPLE 4C

### N-Isopropyl-1-[3-(1-oxido-4-pyridinylethynyl)phenyl]-1,4-dihydro[1,8]naphthyridin-4-one-3-carboxamide

To a solution of N-Isopropyl-1-[3-(4-pyridinylethynyl)phenyl]-1,4-dihydro[1,8]naphthyridin-4-one-3-carboxamide from EXAMPLE 3C in methylene chloride (36 mL/mmol) and methanol (3 mL/mmol) was added magnesium monoperoxyphthalate hexahydrate (MMPP, 3.6 eq) and the mixture was stirred at room temperature overnight. A further amount of MMPP (2 eq) was added and stirring was continued for 24 hours. The resulting mixture was filtered through a bed of celite, the filtrate was diluted with methylene chloride and washed with aqueous sodium bicarbonate and water. After drying, the organic phase was evaporated and the crude product was purified by chromatography on silica gel eluting with 10% methanol in methylene chloride to afford the title compound as-a solid. 1H NMR (CDCl₃) δ 1.28 (d, 6H), 4.28 (m, 1H), 7.35 (d, 2H), 7.46 (m, 2H), 7.58 (m, 2H), 7.67 (m, 1H), 8.14 (d, 2H), 8.69 (m, 1H), 8.81 (dd, 1H), 8.99 (s, 1H), 9.62 (br, NH).

### EXAMPLE 5C

### N-Isopropyl-1-(3-ethynylphenyl)-1,4-dihydro[1,8]naphthyridin-4-one-3-carboxamide

Step 1: N-Isopropyl-1-[3-(trimethylsilylethynyl)phenyl]-1,4-dihydro[1,8]naphthyridin-4-one-3-carboxamide.

Following the procedure of Step 5 of EXAMPLE 1C, but substituting trimethylsilylacetylene for phenylacetylene, the N-isopropyl-1-[3-(trimethylsilylethynyl)phenyl]-1,4-dihydro[1,8]naphthyridin-4-one-3-carboxamide product was obtained and used in the next step without further purification.

Step 2: N-Isopropyl-1-(3-ethynylphenyl)-1,4-dihydro[1,8]naphthyridin-4-one-3-carboxamide.

The crude product from Step 1 was dissolved in methanol (12 mL/mmol) and 1N aqueous sodium hydroxide was added (3 eq), resulting in a suspension. The suspension mixture was stirred at room temperature for 2 hours and the methanol was evaporated. The resulting aqueous suspension was diluted with water and the product was extracted out with ethyl acetate. The crude product was chromatographed on silica gel eluting with 10% ether in methylene chloride to afford the N-isopropyl-1-(3-ethynylphenyl)-1,4-dihydro[1,8]naphthyridin-4-one-3-carboxamide compound as a solid. 1H NMR (Acetone-d₆) □ 1.24 (d, 6H), 3.81 (s, 1H), 4.17 (m, 1H), 7.59 (m, 1H), 7.64-7.71(m, 3H), 7.81 (s, 1H), 8.72 (m, 1H), 8.76 (dd, 1H), 8.84 (s, 1H), 9.61 (br, NH).

### EXAMPLE 6C

### N-Cyclopropyl-1-(3-ethynylphenyl)-1,4-dihydro[1,8]naphthyridin-4-one-3-carboxamide

Step 1: N-Cyclopropyl-1-(3-bromophenyl)-1,4-dihydro[1,8]naphthyridin-4-one-3-carboxamide.

Following the procedure of Step 4 of EXAMPLE 1C, but substituting cyclopropylamine for isopropylamine, the N-cyclopropyl-1-(3-bromophenyl)-1,4-dihydro[1,8]naphthyridin-4-one-3-carboxamide was obtained as a fluffy white solid. 1H NMR (Acetone-d₆) δ 0.59 (m, 2H), 0.80 (m, 2h), 2.96 (m, 1H), 7.59 -7.68 (m, 2H), 7.72 (dd, 1H), 7.82 (dd, 1H), 7.97 (s, 1H), 8.72-8.81 (m, 2H), 8.89 (s, 1H), 9.70 (br, NH).

Steps 2 and 3: N-Cyclopropyl-1-(3-ethynylphenyl)-1,4-dihydro[1,8]naphthyridin-4-one-3-carboxamide

Following the procedures of Steps 1 and 2 of EXAMPLE 5C, but substituting the product from step 1 for N-isopropyl-1-(3-bromophenyl)-1,4-dihydro[1,8]naphthyridin-4-one-3-carboxamide, the N-Cyclopropyl-1-(3-ethynylphenyl)-1,4-dihydro[1,8]naphthyridin-4-one-3-carboxamide compound was obtained as a solid. 1H NMR (CDCl₃) δ 0.66 (m, 2H), 0.85 (m, 2H), 2.97 (m, 1H), 3.18 (s, 1H), 7.42 (d, 1H), 7.47 (m, 1H), 7.52-7.58 (m, 2H), 7.65 (d, 1H), 8.70 (m, 1H), 8.80 (dd, 1H), 8.98 (s, 1H), 9.74 (br, NH).

### EXAMPLE 7C

### N-Isopropyl-1-[3-(3-pyridinylethynyl)phenyl]-1,4-dihydro[1,8]naphthyridin-4-one-3-carboxamide

Following the procedure of Step 5 of EXAMPLE 1C, but substituting N-isopropyl-1-(3-ethynylphenyl)-1,4-dihydro[1,8]naphthyridin-4-one-3-carboxamide from EXAMPLE 5 for phenylacetylene and 3-bromopyridine for N-isopropyl-1-(3-bromophenyl)-1,4-dihydro[1,8]naphthyridin-4-one-3-carboxamide, the title compound was obtained as a light brown solid. 1H NMR (Acetone-d₆) δ 1.24 (d, 6H), 4.18 (m, 1H), 7.43 (m, 1H), 7.61 (m, 1H), 7.70-7.75 (m, 2H), 7.80 (d, 1H), 7.90 (s, 1H), 7.94 (d, 1H), 8.58 (m, 1H), 8.74-8.79 (m, 3H), 8.88 (s, 1H), 9.62 (br, NH).

### EXAMPLE 8C

### N-Isopropyl-1-[3-(1-oxido-3-pyridinylethynyl)phenyl]-1,4-dihydro[1,8]naphthyridin-4-one-3-carboxamide

Following the procedure of EXAMPLE 4C, but substituting N-isopropyl-1-[3-(3-pyridinylethynyl)phenyl]-1,4-dihydro[1,8]naphthyridin-4-one-3-carboxamide from EXAMPLE 7C for N-isopropyl-1-[3-(4-pyridinylethynyl)phenyl]-1,4-dihydro[1,8]naphthyridin-4-one-3-carboxamide, the title compound was obtained as a solid. 1H NMR (CDCl₃) δ 1.28 (d, 6H), 4.28 (m, 1H), 7.26(m, 1H), 7.36 (d, 1H), 7.45-7.49 (m, 2H), 7.57-7.62 (m, 2H), 7.69 (d, 1H), 8.16 (d, 1H), 8.31 (s, 1H), 8.69 (m, 1H), 8.81 (dd, 1H), 8.99 (s, 1H), 9.63 (br, NH).

### EXAMPLE 9C

### N-Cyclopropyl-1-[3-(3-pyridinylethynyl)phenyl]-1,4-dihydro[1,8]naphthyridin-4-one-3-carboxamide

Following the procedure of Step 5 of EXAMPLE 1C, but substituting N-cyclopropyl-1-(3-ethynylphenyl)-1,4-dihydro[1,8]naphthyridin-4-one-3-carboxamide from EXAMPLE 6 for phenylacetylene and 3-bromopyridine for N-isopropyl-1-(3-bromophenyl)-1,4-dihydro[1,8]naphthyridin-4-one-3-carboxamide, the title compound was obtained as a solid. 1H NMR (CDCl₃) δ 0.66 (m, 2H), 0.85 (m, 2H), 2.97 (m, 1H), 7.28 (m, 1H), 7.43-7.48 (m, 2H), 7.57 (t, 1H), 7.62 (s, 1H), 7.70 (d, 1H), 7.79 (d, 1H), 8.55 (m, 1H), 8.70 (m, 1H), 8.75 (s, 1H), 8.79 (dd, 1H), 9.01 (s, 1H), 9.74 (br, NH).

### EXAMPLE 10C

### N-Isopropyl-1-[3-(3-hydroxy-3-methylbut-1-ynyl)phenyl]-1,4-dihydro[1,8]naphthyridin-4-one-3-carboxamide

Following the procedure of Step 5 of EXAMPLE 1C, but substituting 2-methyl-3-butyn-2-ol for phenylacetylene, the title compound was obtained as a white solid. 1H NMR (Acetone-d₆) δ 1.24 (d, 6H), 1.53 (s, 6H), 4.17 (m, 1H), 4.52 (s, 1H, OH), 7.58-7.65 (m, 4H), 7.68 (s, 1H), 8.72 (m, 1H), 8.77 (dd, 1H), 8.84 (s, 1H), 9.62 (br, NH).

### EXAMPLE 11C

### N-Cyclopropyl-1-[3-(3-hydroxy-3-methylbut-1-ynyl)phenyl]-1,4-dihydro[1,8]naphthyridin-4-one-3-carboxamide

Following the procedure of EXAMPLE 10C, but substituting N-cyclopropyl-1-(3-ethynylphenyl)-1,4-dihydro[1,8]naphthyridin-4-one-3-carboxamide from EXAMPLE 6C for N-isopropyl-1-(3-ethynylphenyl)-1,4-dihydro[1,8]naphthyridin-4-one-3-carboxamide, the title compound was obtained as a white solid. 1H NMR (Acetone-d₆) δ 0.57 (m, 2H), 0.78 (m, 2H), 1.53 (s, 6H), 2.93 (m, 1H), 4.53 (s, 1H, OH), 7.58-7.65 (m, 4H), 7.67 (s, 1H), 8.72 (m, 1H), 8.76 (dd, 1H), 8.85 (s, 1H), 9.69 (br, NH).

### EXAMPLE 12C

### N-Isopropyl-1-[3-(1-hydroxycyclopentyl)ethynylphenyl]-1,4-dihydro[1,8]naphthyridin-4-one-3-carboxamide

Following the procedure of Step 5 of EXAMPLE 1C, but substituting 1-ethynylcyclopentanol for phenylacetylene, the title compound was obtained as a white solid. 1H NMR (CDCl₃) δ 1.28 (d, 6H), 1.76-1.80 (m, 2H), 1.84-1.88 (m, 3H), 1.98-2.06 (m, 4H), 4.27 (m, 1H), 7.36 (d, 1H), 7.44-7.50 (m, 3H), 7.56 (d, 1H), 8.68 (m, 1H), 8.79 (dd, 1H), 8.97 (s, 1H), 9.63 (br, NH).

### EXAMPLE 13C

### N-Isopropyl-1-[3-(1-hydroxycyclopropyl)ethynylphenyl]-1,4-dihydro[1,8]naphthyridin-4-one-3-carboxamide

Step 1: 1-Ethynylcyclopropanol.

The 1-ethynylcyclopropanol was prepared following the procedure described in J. Org. Chem. 1976, 41, 1237 from [(1-ethoxycyclopropyl)oxy]trimethylsilane and ethynyl magnesium bromide and was obtained as a liquid.

Step 2: N-Isopropyl-1-[3-(1-hydroxycyclopropyl)ethynylphenyl]-1,4-dihydro[1,8]naphthyridin-4-one-3-carboxamide.

Following the procedure of Step 5 of EXAMPLE 1C, but substituting the product from present Step 1 for phenylacetylene, the N-isopropyl-1-[3-(1-hydroxycyclopropyl)ethynylphenyl]-1,4-dihydro[1,8]naphthyridin-4-one-3-carboxamide compound was obtained as a solid. 1H NMR (CDCl₃) δ 1.09 (m, 2H), 1.17 (m, 2H), 1.28 (d, 6H), 2.57 (s, 1H, OH), 4.28 (m, 1H), 7.35 (d, 1H), 7.44-7.50 (m, 3H), 7.54 (d, 1H), 8.68 (m, 1H), 8.79 (dd, 1H), 8.96 (s, 1H), 9.63 (br, NH).

### EXAMPLE 14C

### N-Isopropyl-1-{3-[4,4,4-trifluoro-3-hydroxy-3-(trifluoromethyl)but-1-ynyl]phenyl}-1,4-dihydro[1,8]naphthyridin-4-one-3-carboxamide

Step 1: 1,1,1-trifluoro-2-(trifluoromethyl)-4-(trimethylsilyl)but-3-yn-2-ol.

To a solution of trimethylsilylacetylene (4 mL) in THF (30 mL) at −78° C. was added 2.5M n-butyllithium in hexanes (14 mL) and the resulting mixture was stirred for 1 hour. An excess of hexafluoroacetone was bubbled into the cold mixture and stirring was continued for 4 hours. After warming to room temperature, the mixture was quenched with saturated aqueous ammonium chloride solution and partitioned between ether and water. The organic phase was dried and evaporated to afford the 1,1,1-trifluoro-2-(trifluoromethyl)4-(trimethylsilyl)but-3-yn-2-ol as a liquid.

Step 2: N-Isopropyl-1-{3-[4,4,4-trifluoro-3-hydroxy-3-(trifluoromethyl)but-1-ynyl]phenyl}-1,4-dihydro[1,8]naphthyridin-4-one-3-carboxamide.

To a solution of 1,1,1-trifluoro-2-(trifluoromethyl)-4-(trimethylsilyl)but-3-yn-2-ol from present Step 1 (6.8 mmol) in 10 mL of THF was added 1M tetrabutylammonium fluoride (8.5 mL) and the resulting mixture was refluxed for 30 minutes to remove the TMS protecting group. The procedure of Step 5 of EXAMPLE 1C was then applied, but substituting this solution for phenylacetylene to afford the N-Isopropyl-1-{3-[4,4,4-trifluoro-3-hydroxy-3-(trifluoromethyl)but-1-ynyl]phenyl}-1,4-dihydro[1,8]naphthyridin-4-one-3-carboxamide compound as a solid. 1H NMR (Acetone-d₆) δ 1.24 (d, 6H), 4.17 (m, 1H), 7.60 (m, 1H), 7.72-7.79 (m, 2H), 7.83 (d, 1H), 7.90 (s, 1H), 8.14 (s, 1H, OH), 8.72 (m, 1H), 8.77 (dd, 1H), 8.85 (s, 1H), 9.62 (br, NH).

### EXAMPLE 15C

### N-Isopropyl-1-[3-(3-hydroxy-3-phenylbut-1-ynyl)phenyl]-1,4-dihydro[1,8]naphthyridin-4-one-3-carboxamide

A mixture of N-isopropyl-1-(3-bromophenyl)-1,4-dihydro[1,8]naphthyridin-4-one-3-carboxamide from Step 4 of EXAMPLE 1C, 2-phenyl-3-butyn-2-ol (2 eq), triethylamine (1.66 eq), bis(diphenylphosphino)ferrocene]dichloropalladium(II) (0.05 eq), and copper (I) iodide (5 mg/mmol) in DMF (20 mL/mmol) was heated at 85° C. for 18 hours. After cooling to room temperature, the resulting mixture was quenched with saturated aqueous ammonium chloride solution and partitioned between ethyl acetate and water. The crude product from the organic phase was chromatographed on silica gel eluting with 20% ether in methylene chloride. The purified product was stirred in ether at room temperature for 3 hours and filtered to afford the title compound as a white solid. 1H NMR (Acetone-d₆) δ 1.24 (d, 6H), 1.79 (s, 3H), 4.18 (m, 1H), 5.22 (s, 1H, OH), 7.26 (t, 1H), 7.35 (t, 2H), 7.59 (m, 1H), 7.66 (m, 3H), 7.73 (d, 2H), 7.76 (s, 1H), 8.72 (m, 1H), 8.77 (dd, 1H), 8.84 (s, 1H), 9.62 (br, NH).

### EXAMPLE 16C

### N-Cyclopropyl-1-[3-(1-oxido-3-pyridinylethynyl)phenyl]-1,4-dihydro[1,8]naphthyridin-4-one-3-carboxamide

Step 1: 3-Ethynylpyridine N-oxide.

To a solution of 3-ethynylpyridine in methylene chloride (5 mL/mmol) at room temperature was added m-chloroperoxybenzoic acid (m-CPBA, 70% purity, 1.2 eq) and the resulting mixture was stirred for 2 hours. A further amount of m-CPBA was added (0.25 eq) and stirring was continued for 1 hour. Calcium hydroxide was added (2 eq) and after 15 minutes the mixture was filtered through celite and the filtrate was evaporated. The solid residue was stirred in ether for 3 hours and filtered to afford the 3-ethynylpyridine N-oxide compound as a white solid.

Step 2: N-Cyclopropyl-1-[3-(1-oxido-3-pyridinylethynyl)phenyl]-1,4-dihydro[1,8]naphthyridin-4-one-3-carboxamide.

Following the procedure of EXAMPLE 15C, but substituting N-cyclopropyl-1-(3-bromophenyl)-1,4-dihydro[1,8]naphthyridin-4-one-3-carboxamide from Step 1 of EXAMPLE 6C for N-isopropyl-1-(3-bromophenyl)-1,4-dihydro[1,8]naphthyridin-4-one-3-carboxamide, and 3-ethynylpyridine N-oxide from Step 1 for 2-phenyl-3-butyn-2-ol, the N-cyclopropyl-1-[3-(1-oxido-3-pyridinylethynyl)phenyl]-1,4-dihydro[1,8]naphthyridin-4-one-3-carboxamide compound was obtained as a white solid. 1H NMR (CDCl₃) δ 0.66 (m, 2H), 0.84 (m, 2H), 2.96 (m, 1H), 7.26 (m, 1H), 7.37 (d, 1H), 7.45-7.48 (m, 2H), 7.58-7.62 (m, 2H), 7.69 (d, 1H), 8.16 (d, 1H), 8.31 (s, 1H), 8.69 (m, 1H), 8.79 (dd, 1H), 8.99 (s, 1H), 9.73 (br, NH).

### EXAMPLE 17C

### N-Isopropyl-1-[3-(3-amino-3-ethylpent-1-ynyl)phenyl]-1,4-dihydro[1,8]naphthyridin-4-one-3-carboxamide

Following the procedure of EXAMPLE 15C, but substituting 1,1-diethylpropargylamine for 2-phenyl-3-butyn-2-ol, the title compound was obtained as a solid. 1H NMR (CDCl₃) δ 1.05 (t, 6H), 1.28 (d, 6H), 1.57 (m, 2H), 1.69 (m, 2H), 4.27 (m, 1H), 7.33 (d, 1H), 7.44-7.49 (m, 3H), 7.53 (d, 1H), 8.69 (m, 1H), 8.79 (dd, 1H), 8.97 (s, 1H), 9.63 (br, NH). (NH₂ not observed).

### EXAMPLE 18C

### N-Cyclopropyl-1-[3-(3-amino-3-ethylpent-1-ynyl)phenyl]-1,4-dihydro[1,8]naphthyridin-4-one-3-carboxamide

Following the procedure of EXAMPLE 17C, but substituting N-cyclopropyl-1-(3-bromophenyl)-1,4-dihydro[1,8]naphthyridin-4-one-3-carboxamide from Step 1 of EXAMPLE 6C for N-isopropyl-1-(3-bromophenyl)-1,4-dihydro[1,8]naphthyridin-4-one-3-carboxamide, the title compound was obtained as a solid. 1H NMR (CDCl₃) δ 0.66 (m, 2H), 0.84 (m, 2H), 1.05 (t, 6H), 1.57 (m, 2H), 1.70 (m, 2H), 2.96 (m, 1H), 7.33 (d, 1H), 7.44-7.49 (m, 3H), 7.54 (d, 1H), 8.69 (m, 1H), 8.77 (dd, 1H), 8.97 (s, 1H), 9.75 (br, NH). (NH₂ not observed).

### EXAMPLE 19C

### N-Isopropyl-1-[3-(3-quinolinylethynyl)phenyl]-1,4-dihydro[1,8]naphthyridin-4-one-3-carboxamide

Following the procedure of EXAMPLE 15C, but substituting N-isopropyl-1-(3-ethynylphenyl)-1,4-dihydro[1,8]naphthyridin-4-one-3-carboxamide from Step 2 of EXAMPLE 5C for 2-phenyl-3-butyn-2-ol, and 3-bromoquinoline for N-isopropyl-1-(3-bromophenyl)-1,4-dihydro[1,8]naphthyridin-4-one-3-carboxamide, the title compound was obtained as a solid. 1H NMR (CDCl₃) δ 1.32 (d, 6H), 4.32 (m, 1H), 7.48-7.51 (m, 2H), 7.58-7.65 (m, 2H), 7.71 (s, 1H), 7.73-7.80 (m, 2H), 7.83 (d, 1H), 8.12 (d, 1H), 8.35 (s, 1H), 8.75 (m, 1H), 8.85 (dd, 1H), 9.02 (s, 1H), 9.06 (s, 1H), 9.65 (br, NH).

### EXAMPLE 20C

### N-Isopropyl-1-[3-(1-oxido-3-quinolinylethynyl)phenyl]-1,4-dihydro[1,8]naphthyridin-4-one-3-carboxamide

Following the procedure of EXAMPLE 19C, but substituting 3-bromoquinoline N-oxide for 3-bromoquinoline, the title compound was obtained as a solid. 1H NMR (CDCl₃) δ 1.33 (d, 6H), 4.32 (m, 1H), 7.49-7.53 (m, 2H), 7.63 (t, 1H), 7.68-7.73 (m, 2H), 7.75-7.83 (m, 2H), 7.88-7.92 (m, 2H), 8.63 (s, 1H), 8.73-8.78 (m, 2H), 8.86 (dd, 1H), 9.05 (s, 1H), 9.67 (br, NH).

### EXAMPLE 21C

### N-Isopropyl-1-[3-(cyclopropylethynyl)phenyl]-1,4-dihydro[1,8]naphthyridin-4-one-3-carboxamide

Following the procedure of EXAMPLE 15C, but substituting cyclopropylacetylene (Tetrahedron letters 2000, 41, 4007) for 2-phenyl-3-butyn-2-ol, the title compound was obtained as a gray solid. 1H NMR (CDCl₃) δ 0.83 (m, 2H), 0.90 (m, 2H), 1.31 (d, 6H), 1.48 (m, 1H), 4.31 (m, 1H), 7.33 (m, 1H), 7.45-7.51 (m, 3H), 7.55 (d, 1H), 8.72 (m, 1H), 8.83 (dd, 1H), 9.01 (s, 1H), 9.68 (br, NH).

### EXAMPLE 22C

### N-Isopropyl-1-[3-(6-amino-3-pyridinylethynyl)phenyl]-1,4-dihydro[1,8]naphthyridin-4-one-3-carboxamide

Following the procedure of EXAMPLE 19C, but substituting but substituting 5-bromo-2-aminopyridine for 3-bromoquinoline, the title compound was obtained as a solid. 1H NMR (CDCl₃) δ 1.33 (d, 6H), 4.31 (m, 1H), 4.71 (br, NH₂), 6.49 (d, 1H), 7.40 (m, 1H), 7.48 (m, 1H), 7.54-7.60 (m, 3H), 7.68 (d, 1H), 8.28 (s, 1H), 8.72 (m, 1H), 8.83 (dd, 1H), 9.04 (s, 1H), 9.67 (br, NH).

### EXAMPLE 23C

### N-Isopropyl-1-{3-[5-(1-hydroxy-1-methylethyl)-1-oxido-3-pyridinylethynyl]phenyl}-1,4-dihydro[1,8]naphthyridin-4-one-3-carboxamide

Step 1: 3-Bromo-5-(1-hydroxy-1-methylethyl)pyridine.

To a solution of ethyl 5-bromonicotinate (1.02 g, 4.4 mmol) in diethyl ether (15 mL) at −30° C. was added a 3M solution of methyl magnesium bromide in ether (4 mL, 12 mmol). The resulting slurry was refluxed for 2 hours then cooled and quenched with an excess of 0.5M aqueous monobasic sodium phosphate and partitioned between ether and water. The product from the organic phase was chromatographed on silica gel eluting with a 2:1:2 mixture of ether, pentane and ammonia-saturated methylene chloride to afford the 3-bromo-5-(1-hydroxy-1-methylethyl)pyridine compound as a yellow oil.

Step 2: 3-Bromo-5-(1-hydroxy-1-methylethyl)pyridine-N-oxide.

To a solution of 3-bromo-5-(1-hydroxy-1-methylethyl)pyridine from Step 1 (3.1 mmol) in chloroform (10 mL) was added m-chloroperoxybenzoic acid 70% (1.5 eq) and the resulting mixture was stirred at room temperature for 18 hours. An excess of calcium hydroxide was added and after stirring for 5 minutes, the mixture was filtered through celite and the filtrate was evaporated. The crude material was chromatographed on silica gel eluting with 10% ethanol in methylene chloride (saturated with ammonia) to afford the 3-bromo-5-(1-hydroxy-1-methylethyl)pyridine-N-oxide compound as a solid.

Step 3: N-Isopropyl-1-{3-[5-(1-hydroxy-1-methylethyl)-1-oxido-3-pyridinylethynyl]phenyl}-1,4-dihydro[1,8]naphthyridin-4-one-3-carboxamide.

Following the procedure of EXAMPLE 15C, but substituting N-isopropyl-1-(3-ethynylphenyl)-1,4-dihydro[1,8]naphthyridin-4-one-3-carboxamide from EXAMPLE 5C for 2-phenyl-3-butyn-2-ol, and 3-bromo-5-(1-hydroxy-1-methylethyl)pyridine-N-oxide from Step 2 for N-isopropyl-1-(3-bromophenyl)-1,4-dihydro[1,8]naphthyridin-4-one-3-carboxamide, the N-isopropyl-1-{3-[5-(1-hydroxy-1-methylethyl)-1-oxido-3-pyridinylethynyl]phenyl}-1,4-dihydro[1,8]naphthyridin-4-one-3-carboxamide compound was obtained as a solid. 1H NMR (CDCl₃) δ 1.32 (d, 6H), 1.64 (s, 6H), 2.22 (br, 1H, OH), 4.30 (m, 1H), 7.45-7.52 (m, 2H), 7.60 (t, 1H), 7.66 (s, 1H), 7.72 (d, 1H), 7.98 (s, 1H), 8.70 (br, 2H), 8.73 (m, 1H), 8.84 (dd, 1H), 9.03 (s, 1H), 9.68 (br, NH).

### EXAMPLE 24C

### N-Isopropyl-1-{3-[6-(1-hydroxy-1-methylethyl)-3-pyridinylethynyl]phenyl}-1,4-dihydro[1,8]naphthyridin-4-one-3-carboxamide

Step 1: 5-Bromo-2-(1-hydroxy-1-methylethyl) pyridine.

To a suspension of 2,5-dibromopyridine in toluene (12 mL/mmol) cooled to −78° C. was added n-butyllithium 2.5M in hexanes (1.05 eq) and the resulting mixture was stirred in the cold for 2.5 hours. Acetone (2 eq) was added and stirring was continued for 1.5 h. After quenching with saturated aqueous ammonium chloride solution, the mixture was warmed to room temperature and partitioned between ethyl acetate and water. The product from the organic phase was chromatographed on silica gel eluting with 20% ethyl acetate in hexane to afford the 5-bromo-2-(1-hydroxy-1-methylethyl) pyridine compound as a syrup.

Step 2: 5-Bromo-2-(1-methyl-1-{[2-(trimethylsilyl)ethoxy]methoxy}ethyl)pyridine.

To a solution of 5-bromo-2-(1-hydroxy-1-methylethyl) pyridine from Step 1 (14 mmol) in methylene chloride (50 mL) at 0° C. was added N,N-diisopropylethylamine (37.3 mmol) and 2-(trimethylsilyl)ethoxymethyl chloride (15.3 mmol). The resulting mixture was stirred at room temperature for 18 hours, then refluxed for 24 hours. After cooling to room temperature the mixture was quenched with saturated aqueous ammonium chloride solution and partitioned between methylene chloride and water. The crude product from the organic phase was chromatographed on silica gel eluting with 6% ethyl acetate in hexane to afford the 5-bromo-2-(1-methyl-1-{[2-(trimethylsilyl)ethoxy]methoxy}ethyl)pyridine compound.

Step 3: 2-(1-Methyl-1-{[2-(trimethylsilyl)ethoxy]methoxy}ethyl)-5-[(trimethylsilyl)ethynyl]pyridine.

Following the procedure of Step 5 of EXAMPLE 1C, but substituting the product from present Step 2 for N-isopropyl-1-(3-bromophenyl)-1,4-dihydro[1,8]naphthyridin-4-one-3-carboxamide and trimethylsilylacetylene for phenylacetylene, the 2-(1-methyl-1-{[2-(trimethylsilyl)ethoxy]methoxy}ethyl)-5-[(trimethylsilyl)ethynyl]pyridine compound was obtained.

Step 4: 5-Ethynyl-2-(1-methyl-1-{[2-(trimethylsilyl)ethoxy]methoxy}ethyl)pyridine.

Following the procedure of Step 2 of EXAMPLE 5C, but substituting the product from present Step 3 for N-isopropyl-1-[3-(trimethylsilylethynyl)phenyl]-1,4-dihydro[1,8]naphthyridin-4-one-3-carboxamide, the 5-ethynyl-2-(1-methyl-1-{[2-(trimethylsilyl)ethoxy]methoxy}ethyl)pyridine compound was obtained.

Step 5: N-Isopropyl-1-(3-{[6-(1-methyl-1-{{2-trimethylsilyl)ethoxy]methoxy}ethyl)pyridin-3-yl]ethynyl}phenyl)-1,4-dihydro[1,8]naphthyridin-4-one-3-carboxamide.

Following the procedure of Step 5 of EXAMPLE 1C, but substituting the product from present Step 4 for phenylacetylene, the N-isopropyl-1-(3-{[6-(1-methyl-1-{{2-trimethylsilyl)ethoxy]methoxy}ethyl)pyridin-3-yl]ethynyl}phenyl)-1,4-dihydro[1,8]naphthyridin-4-one-3-carboxamide compound was obtained.

Step 6: N-Isopropyl-1-{3-[6-(1-hydroxy-1-methylethyl)-3-pyridinylethynyl]phenyl}-1,4-dihydro[1,8]naphthyridin-4-one-3-carboxamide.

To a solution of N-isopropyl-1-(3-{[6-(1-methyl-1-{{2-trimethylsilyl)-ethoxy]methoxy}ethyl)pyridin-3-yl]ethynyl}phenyl)-1,4-dihydro[1,8]naphthyridin-4-one-3-carboxamide product from present Step 5 in methylene chloride (32 mL/mmol) at 0° C. was added trifluoroacetic acid (3.2 mL/mmol). The resulting mixture was stirred at 0° C. for 2 hours then at room temperature for 2 hours. The mixture was neutralized slowly with saturated aqueous sodium bicarbonate and partitioned between methylene chloride and water. The crude material from the organic phase was chromatographed on silica gel eluting with 40% ether in methylene chloride and the purified product was stirred in ether at room temperature for 2 hours and filtered to afford the N-isopropyl-1-{3-[6-(1-hydroxy-1-methylethyl)-3-pyridinylethynyl]phenyl)}-1,4-dihydro[1,8]naphthyridin-4-one-3-carboxamide compound as solid. 1H NMR (Acetone-d₆) δ 1.24 (d, 6H), 1.50 (s, 6H), 4.18 (m, 1H), 4.57 (s, 1H, OH), 7.61 (m, 1H), 7.69-7.74 (m, 3H), 7.78 (m, 1H), 7.88 (s, 1H), 7.93 (dd, 1H), 8.68 (s, 1H), 8.74 (m, 1H), 8.78 (dd, 1H), 8.88 (s, 1H), 9.63 (br, NH).

### EXAMPLE 25C

### N-Isopropyl-1-{3-[6-(1-hydroxy-1-methylethyl)-1-oxido-3-pyridinylethynyl]phenyl}-1,4-dihydro[1,8]naphthyridin-4-one-3-carboxamide

Following the procedure of Step 2 of EXAMPLE 23C, but substituting N-isopropyl-1-{3-[6-(1-hydroxy-1-methylethyl)-3-pyridinylethynyl]phenyl}-1,4-dihydro[1,8]naphthyridin-4-one-3-carboxamide from example 24C for 3-bromo-5-(1-hydroxy-1-methylethyl)pyridine, the title compound was obtained as a solid. 1H NMR (Acetone-d₆) δ 1.25 (d, 6H), 1.60 (s, 6H), 4.18 (m, 1H), 7.24 (s, 1H, OH), 7.60-7.63 (m, 3H), 7.72-7.78 (m, 2H), 7.82 (d, 1H), 7.91 (s, 1H), 8.46 (s, 1H), 8.74 (m, 1H), 8.78 (dd, 1H), 8.87 (s, 1H), 9.62 (br, NH).

### EXAMPLE 26C

### N-Isopropyl-1-{3-[4-(1-hydroxy-1-methylethyl)-2-pyridinylethynyl]phenyl}-1,4-dihydro[1,8]naphthyridin-4-one-3-carboxamide

Step 1: Methyl 2-bromoisonicotinate.

To a solution of 2-bromoisonicotinic acid (Chem. Pharm. Bull. 1990, 38, 2446) (2.0 g) in tetrahydrofuran (100 mL) was added excess ethereal diazomethane and the resulting mixture was stirred at room temperature for 1 hour. The mixture was evaporated and the product chromatographed on silica gel eluting with a 1:3 mixture of ethyl acetate and hexane to afford the methyl 2-bromoisonicotinate ester as a colorless liquid.

Step 2: 2-Bromo-4-(1-hydroxy-1-methylethyl)pyridine.

Following the procedure of Step 1 of EXAMPLE 23C, but substituting methyl 2-bromoisonicotinate from present Step 1 for ethyl 5-bromonicotinate, the 2-bromo-4-(1-hydroxy-1-methylethyl)pyridine compound was obtained as a white solid.

Step 3: N-Isopropyl-1-{3-[4-(1-hydroxy-1-methylethyl)-2-pyridinylethynyl]phenyl}-1,4-dihydro[1,8]naphthyridin-4-one-3-carboxamide.

Following the procedure of EXAMPLE 19C, but substituting the 2-bromo-4-(1-hydroxy-1-methylethyl)pyridine from present Step 2 for 3-bromoquinoline, the N-isopropyl-1-{3-[4-(1-hydroxy-1-methylethyl)-2-pyridinylethynyl]phenyl}-1,4-dihydro[1,8]naphthyridin-4-one-3-carboxamide compound was obtained as a yellow foam. 1H NMR (Acetone-d₆) δ 1.27 (d, 6H), 1.55 (s, 6H), 4.20 (m, 1H), 4.42 (s, 1H, OH), 7.52 (m, 1H), 7.63 (m, 1H), 7.72-7.79 (m, 3H), 7.84 (d, 1H), 7.95 (s, 1H); 8.55 (d, 1H), 8.77 (m, 1H), 8.80 (dd, 1H), 8.92 (s, 1H), 9.65 (br, NH).

### EXAMPLE 27C

### N-Isopropyl-1-{3-[5-(1-hydroxy-1-methylethyl)-2-pyridinylethynyl]phenyl}-1,4-dihydro[1,8]naphthyridin-one-3-carboxamide

Step 1: 2-Bromo-5-(1-hydroxy-1-methylethyl)pyridine.

A solution of 2,5-dibromopyridine in diethyl ether (5 mL/mmol) was cooled to −78° C., and n-butyllithium 2.5M in hexanes (1.05 eq) was added slowly. After 2 hrs in the cold, acetone (1.3 eq) was added and stirring was continued for 1 hour. The resulting mixture was quenched with saturated aqueous ammonium chloride solution, warmed to room temperature, and partitioned between ether and water. The crude product from the organic phase was triturated with 1:1 ether-hexane and filtered to afford the 2-bromo-5-(1-hydroxy-1-methylethyl)pyridine compound as a solid.

Step 2: 5-(1-Hydroxy-1-methylethyl)-2-[(trimethylsilyl)ethynyl]pyridine.

Following the procedure of EXAMPLE 15C, but substituting the product 2-bromo-5-(1-hydroxy-1-methylethyl)pyridine from present Step 1 for N-isopropyl-1-(3-bromophenyl)-1,4-dihydro[1,8]naphthyridin-4-one-3-carboxamide and trimethylsilylacetylene for 2-phenyl-3-butyn-2-ol, the 5-(1-hydroxy-1-methylethyl)-2-[(trimethylsilyl)ethynyl]pyridine compound was obtained.

Step 3: 2-Ethynyl-5-(1-hydroxy-1-methylethyl)pyridine.

Following the procedure of Step 2 of EXAMPLE 5C, but substituting the product 5-(1-hydroxy-1-methylethyl)-2-[(trimethylsilyl)ethynyl]pyridine from present Step 2 for N-isopropyl-1-[3-(trimethylsilylethynyl)phenyl]-1,4-dihydro[1,8]naphthyridin-4-one-3-carboxamide, the 2-ethynyl-5-(1-hydroxy-1-methylethyl)pyridine compound was obtained.

Step 4: N-Isopropyl-1-{3-[5-(1-hydroxy-1-methylethyl)-2-pyridinylethynyl]phenyl}-1,4-dihydro[1,8]naphthyridin-4-one-3-carboxamide.

Following the procedure of EXAMPLE 15C but substituting the product 2-ethynyl-5-(1-hydroxy-1-methylethyl)pyridine from present Step 3 for 2-phenyl-3-butyn-2-ol, the N-Isopropyl-1-{3-[5-(1-hydroxy-1-methylethyl)-2-pyridinylethynyl]phenyl}-1,4-dihydro[1,8]naphthyridin-4-one-3-carboxamide compound was obtained. 1H NMR (CDCl₃) δ 1.32 (d, 6H), 1.66 (s, 6H), 2.08 (s, 1H, OH), 4.31 (m, 1H), 7.46-7.55 (m, 3H), 7.61 (t, 1H), 7.71 (s, 1H), 7.78 (d, 1H), 7.86 (dd, 1H), 8.73 (m, 1H), 8.77 (m, 1H), 8.83 (dd, 1H), 9.04 (s, 1H), 9.67 (br, NH).

### EXAMPLE 28C

### N-Isopropyl-1-{3-[6-(1-hydroxy-1-methylethyl)-2-pyridinylethynyl]phenyl}-1,4-dihydro[1,8]naphthyridin-4-one-3-carboxamide

Step 1: 2-Bromo-6-(1-hydroxy-1-methylethyl)pyridine.

Following the procedure of Step 1 of EXAMPLE 27C, but substituting 2,6-dibromopyridine for 2,5-dibromopyridine, the 2-Bromo-6-(1-hydroxy-1-methylethyl)pyridine compound was obtained as a solid.

Step 2: N-Isopropyl-1-{3-[6-(1-hydroxy-1-methylethyl)-2-pyridinylethynyl]phenyl}-1,4-dihydro[1,8]naphthyridin-4-one-3-carboxamide.

Following the procedure of EXAMPLE 19C, but substituting the product 2-bromo-6-(1-hydroxy-1-methylethyl)pyridine from present Step 1 for 3-bromoquinoline, the N-Isopropyl-1-{3-[6-(1-hydroxy-1-methylethyl)-2-pyridinylethynyl]phenyl}-1,4-dihydro[1,8]naphthyridin-4-one-3-carboxamide compound was obtained. 1H NMR (CDCl₃) δ 1.31 (d, 6H), 1.58 (s, 6H), 4.32 (m, 1H), 4.83 (s, 1H, OH), 7.38 (d, 1H), 7.43-7.52 (m, 3H), 7.60 (t, 1H), 7.70-7.75 (m, 2H), 7.79 (d, 1H), 8.74 (m, 1H), 8.84 (dd, 1H), 9.03 (s, 1H), 9.66 (br, NH).

### EXAMPLE 29C

### N-Cyclopropyl-1-{3-[6-(1-hydroxy-1-methylethyl)-1-oxido-3-pyridinylethynyl]phenyl}-1,4-dihydro[1,8]naphthyridin-4-one-3-carboxamide

Step 1: 5-Bromo-2-(1-hydroxy-1-methylethyl)pyridine N-oxide.

Following the procedure of Step 2 of EXAMPLE 23C, but substituting 5-bromo-2-(1-hydroxy-1-methylethyl)pyridine from Step 1 of EXAMPLE 24C for 3-bromo-5-(1-hydroxy-1-methylethyl)pyridine, the 5-bromo-2-(1-hydroxy-1-methylethyl)pyridine N-oxide compound was obtained.

Step 2: N-Cyclopropyl-1-{3-[6-(1-hydroxy-1-methylethyl)-1-oxido-3-pyridinylethynyl]phenyl}-1,4-dihydro[1,8]naphthyridin-4-one-3-carboxamide.

Following the procedure of EXAMPLE 15C, but substituting N-cyclopropyl-1-(3-ethynylphenyl)-1,4-dihydro[1,8]naphthyridin-4-one-3-carboxamide from EXAMPLE 6C for 2-phenyl-3-butyn-2-ol and 5-bromo-2-(1-hydroxy-1-methylethyl)pyridine-N-oxide from present Step 1 for N-isopropyl-1-(3-bromophenyl)-1,4-dihydro[1,8]naphthyridin-4-one-3-carboxamide, the N-cyclopropyl-1-{3-[6-(1-hydroxy-1-methylethyl)-1-oxido-3-pyridinylethynyl]phenyl}-1,4-dihydro[1,8]naphthyridin-4-one-3-carboxamide compound was obtained as a solid. 1H NMR (CDCl₃) δ 0.66 (m, 2H), 0.84 (m, 2H), 1.66 (s, 6H), 2.96 (m, 1H), 7.34 (d, 1H), 7.43-7.50 (m, 4H), 7.58-7.62 (m, 2H), 7.69 (d, 1H), 8.33 (s, 1H, OH), 8.69 (m, 1H), 8.79 (dd, 1H), 8.99 (s, 1H), 9.73 (br, NH).

### EXAMPLE 30C

### N-Isopropyl-1-{3-[(4-pyridin-3-ylphenyl)ethynyl]phenyl}-1,4-dihydro[1-8]naphthyridin-4-one-3-carboxamide

Step 1: 3-(4-Bromophenyl)pyridine.

A mixture of pyridine-3-boronic acid 1,3-propanediol cyclic ester, 4-bromo-iodobenzene (1.1 eq), [1,1′-bis (diphenylphosphino)ferrocene]dichloropalladium(II) (0.05 eq) and 2M aqueous sodium carbonate (5 eq) in N,N-dimethylformamide (2 mL/mmol) was stirred at 85° C. for 4 hours. After quenching with saturated aqueous ammonium chloride solution the mixture was partitioned between ethyl acetate and water and the crude product from the organic phase was chromatographed on silica gel eluting with a 1:9 mixture of ethyl acetate and hexane to afford the 3-(4-bromophenyl)pyridine compound as a solid.

Step 2: N-Isopropyl-1-{3-[(4-pyridin-3-ylphenyl)ethynyl]phenyl}-1,4-dihydro[1,8]naphthyridin-4-one-3-carboxamide.

Following the procedure of EXAMPLE 19C, but substituting the product from present Step 1 for 3-bromoquinoline, the N-isopropyl-1-{3-[(4-pyridin-3-ylphenyl)ethynyl]phenyl}-1,4-dihydro[1,8]naphthyridin-4-one-3-carboxamide compound was obtained. 1H NMR (CDCl₃) δ 1.28 (d, 6H), 4.28 (m, 1H), 7.38 (m, 1H), 7.42 (d, 1H), 7.48 (m, 1H), 7.53-7.64 (m, 6H), 7.70 (d, 1H), 7.88 (d, 1H), 8.60 (m, 1H), 8.71 (m, 1H), 8.82 (dd, 1H), 8.86 (s, 1H), 9.02 (s, 1H), 9.63 (br, NH).

### EXAMPLE 31C

### N-Isopropyl-1-(3-{[5-(1-hydroxy-1-methylethyl)thien-2-yl]ethynyl}phenyl)-1,4-dihydro[1,8]naphthyridin-4-one-3-carboxamide

Step 1: 2-Bromo-5-(1-hydroxy-1-methylethyl)thiophene.

To a solution of 2-acetyl-5-bromothiophene in THF (2.5 mL/mmol) at −30° C. was added 1.4M methylmagnesium bromide in 3:1 toluene-THF (1.5 eq) and the resulting mixture was warmed to −10° C. and stirred for 1.5 hours. After quenching with saturated aqueous ammonium chloride solution, the mixture was partitioned between ether and water. The organic fraction was dried and evaporated, and the crude material was chromatographed on silica gel eluting with a 1:4 mixture of ether and hexane to afford the 2-bromo-5-(1-hydroxy-1-methylethyl)thiophene compound.

Step 2: 2-(1-Hydroxy-1-methylethyl)-5-trimethylsilylethynyl thiophene.

Following the procedure of EXAMPLE 15C, but substituting the product from present Step 1 for N-isopropyl-1-(3-bromophenyl)-1,4-dihydro[1,8]naphthyridin-4-one-3-carboxamide and trimethylsilylacetylene for 2-phenyl-3-butyn-2-ol, the 2-(1-hydroxy-1-methylethyl)-5-trimethylsilylethynyl thiophene compound was obtained.

Step 3: 2-Ethynyl-5-(1-hydroxy-1-methylethyl)thiophene.

Following the procedure of Step 2 of EXAMPLE 5C, but substituting the product from present Step 2 for N-isopropyl-1-[3-(trimethylsilylethynyl)phenyl]-1,4-dihydro[1,8]naphthyridin-4-one-3-carboxamide, the 2-ethynyl-5-(1-hydroxy-1-methylethyl)thiophene compound was obtained.

Step 4: N-Isopropyl-1-(3-{[5-(1-hydroxy-1-methylethyl)thien-2-yl]ethynyl}phenyl)-1,4-dihydro[1,8]naphthyridin-4-one-3-carboxamide.

Following the procedure of EXAMPLE 15C, but substituting the 2-ethynyl-5-(1-hydroxy-1-methylethyl)thiophene product from present Step 3 for 2-phenyl-3-butyn-2-ol, the N-isopropyl-1-(3-{[5-(1-hydroxy-1-methylethyl)thien-2-yl]ethynyl}phenyl)-1,4-dihydro[1,8]naphthyridin-4-one-3-carboxamide compound was obtained as a solid. 1H NMR (CDCl₃) δ 1.31 (d, 6H), 1.70 (s, 6H), 2.42 (s, 1H, OH), 4.31 (m, 1H), 6.87 (d, 1H), 7.16 (d, 1H), 7.42 (d, 1H), 7.48 (m, 1H), 7.59 (t, 1H), 7.63 (s, 1H), 7.68 (d, 1H), 8.73 (m, 1H), 8.84 (dd, 1H), 9.02 (s, 1H), 9.68 (br, NH).

### EXAMPLE 32C

### N-Isopropyl-1-(3-{[2-(1-hydroxy-1-methylethyl)-1,3-thiazol-5-yl]ethynyl}phenyl)-1,4-dihydro[1,8]naphthyridin-4-one-3-carboxamide

Step 1: 2-(1-hydroxy-1-methylethyl) thiazole.

To a solution of thiazole in ether (1 mL/mmol) at −78° C. was added 2.2M n-butyllithium in hexanes (1.1 eq) and the resulting mixture was stirred for 30 minutes. Acetone (1.2 eq) was added and the mixture was stirred at −78° C. for a further 30 minutes. The mixture was quenched in the cold with saturated aqueous ammonium chloride solution and warmed to room temperature, then partitioned between ether and water. The organic phase was dried and evaporated to yield the crude product as an orange-brown oil which was used as such in the next step.

Step 2: 2-(1-methyl-1-{[2-(trimethylsilyl)ethoxy]methoxy}ethyl)thiazole.

Following the procedure of Step 2 of EXAMPLE 24C, but substituting the product from present Step 1 for 5-bromo-2-(1-hydroxy-1-methylethyl) pyridine, the 2-(1-methyl-1-{[2-(trimethylsilyl)ethoxy]methoxy}ethyl)thiazole compound was obtained as an oil.

Step 3: 5-Bromo-2-(1-hydroxy-1-methylethyl)thiazole.

To a solution of 2-(1-methyl-1-{[2-(trimethylsilyl)ethoxy]methoxy)ethyl)thiazole from Step 2 in chloroform (2 mL/mmol) at room temperature was added bromine (2 molar eq) and the resulting mixture was stirred for 1 hour. Solid sodium bicarbonate (0.55 eq) was added and the mixture was stirred for 5 hours. More sodium bicarbonate was added (0.55 eq) and stirring was continued for 18 hours. After a final addition of sodium bicarbonate (0.55 eq) the mixture was stirred for a further 5 hours, diluted with chloroform and the organic phase was washed with saturated aqueous sodium bicarbonate, then with water, dried and evaporated. The crude material was chromatographed, eluting with a 3:7 mixture of ethyl acetate and hexane to afford the desired product.

Step 4: N-Isopropyl-1-(3-{[2-(1-hydroxy-1-methylethyl)-1,3-thiazol-5-yl]ethynyl}phenyl)-1,4-dihydro[1,8]naphthyridin-4-one-3-carboxamide.

Following the procedure of EXAMPLE 19C, but substituting the product from present Step 3 for 3-bromoquinoline, the N-isopropyl-1-(3-{[2-(1-hydroxy-1-methylethyl)-1,3-thiazol-5-yl]ethynyl}phenyl)-1,4-dihydro[1,8]naphthyridin-4-one-3-carboxamide compound was obtained as a solid. 1H NMR (CDCl₃) δ 1.29 (d, 6H), 1.68 (s, 6H), 2.90 (s, 1H, OH), 4.28 (m, 1H), 7.42 (d, 1H), 7.46 (m, 1H), 7.54-7.60 (m, 2H), 7.66 (d, 1H), 7.82 (s, 1H), 8.70 (m, 1H), 8.80 (dd, 1H), 8.99 (s, 1H), 9.63 (br, NH).

### EXAMPLE 33C

### 1-[3-(1-Oxido-3-pyridinylethynyl)phenyl]-1,4-dihydro[1,8]naphthyridin-4-one-3-carboxamide

Step 1: 1-(3-Bromophenyl)-1,4-dihydro[1,8]naphthyridin-4-one-3-carboxamide.

Following the procedure of Step 4 of EXAMPLE 1C, but substituting 28% aqueous ammonium hydroxide for isopropylamine, the 1-(3-bromophenyl)-1,4-dihydro[1,8]naphthyridin-4-one-3-carboxamide compound was obtained as a solid.

Step 2: 1-[3-(Trimethylsilylethynyl)phenyl]-1,4-dihydro[1,8]naphthyridin-4-one-3-carboxamide.

Following the procedure of Step 5 of EXAMPLE 1C, but substituting the 1-(3-Bromophenyl)-1,4-dihydro[1,8]naphthyridin-4-one-3-carboxamide from present Step 1 for N-isopropyl-1-(3-bromophenyl)-1,4-dihydro[1,8]naphthyridin-4-one-3-carboxamide and trimethylsilylacetylene for phenylacetylene, the 1-[3-(trimethylsilylethynyl)phenyl]-1,4-dihydro[1,8]naphthyridin-4-one-3-carboxamide product was obtained as a solid.

Step 3: 1-(3-Ethynylphenyl)-1,4-dihydro[1,8]naphthyridin-4-one-3-carboxamide.

To a solution of 1-[3-(trimethylsilylethynyl)phenyl]-1,4-dihydro[1,8]naphthyridin-4-one-3-carboxamide from Step 2 in THF (30 mL/mmol) at 0° C. was added 1M tetrabutylammonium fluoride in THF (1.5 eq) and the resulting mixture was stirred at 0° C. for 30 minutes. The mixture was partitioned between methylene chloride and water and the organic phase was dried and evaporated. The crude 1-(3-ethynylphenyl)-1,4-dihydro[1,8]naphthyridin-4-one-3-carboxamide product was used as such in the next step.

Step 4: 1-[3-(1-oxido-3-pyridinylethynyl)phenyl]-1,4-dihydro[1,8]naphthyridin-4-one-3-carboxamide.

Following the procedure of example 19C, but substituting the 1-(3-ethynylphenyl)-1,4-dihydro[1,8]naphthyridin-4-one-3-carboxamide from Step 3 for N-Isopropyl-1-(3-ethynylphenyl)-1,4-dihydro[1,8]naphthyridin-4-one-3-carboxamide and 3-bromopyridine N-oxide for 3-bromoquinoline, the 1-[3-(1-oxido-3-pyridinylethynyl)phenyl]-1,4-dihydro[1,8]naphthyridin-4-one-3-carboxamide was obtained as a white solid. 1H NMR (CDCl₃) δ 5.84 (br, 1H, NH), 7.30 (m, 1H), 7.41 (d, 1H), 7.53 (m, 2H), 7.64 (t, 1H), 7.67 (s, 1H), 7.74 (d, 1H), 8.21 (d, 1H), 8.35 (s, 1H), 8.75 (m, 1H), 8.88 (dd, 1H), 9.05 (s, 1H), 9.52 (br, 1H, NH).

### EXAMPLE 34C

### 1-[3-(1-Oxido-3-pyridinylethynyl)phenyl]-1,4-dihydro[1,8]naphthyridin-4-one-3-carboxylic acid

Step 1: Ethyl 1-(3-Ethynylphenyl)-1,4-dihydro[1,8]naphthyridin-4-one-3-carboxylate.

Following the procedures of Steps 1 and 2 of EXAMPLE 5C, but substituting ethyl 1-(3-bromophenyl)-1,4-dihydro[1,8]naphthyridin-4-one-3-carboxylate from Step 2 of EXAMPLE 1 for N-isopropyl-1-(3-bromophenyl)-1,4-dihydro[1,8]naphthyridin-4-one-3-carboxamide as the starting material, the ethyl 1-(3-Ethynylphenyl)-1,4-dihydro[1,8]naphthyridin-4-one-3-carboxylate compound was obtained as a solid.

Step 2: Ethyl 1-[3-(1-oxido-3-pyridinylethynyl)phenyl]-1,4-dihydro[1,8]naphthyridin-4-one-3-carboxylate.

Following the procedure of EXAMPLE 15C, but substituting the ethyl 1-(3-ethynylphenyl)-1,4-dihydro[1,8]naphthyridin-4-one-3-carboxylate from present Step 1 for 2-phenyl-3-butyn-2-ol and 3-bromopyridine N-oxide for N-isopropyl-1-(3-bromophenyl)-1,4-dihydro[1,8]naphthyridin-4-one-3-carboxamide, the ethyl 1-[3-(1-oxido-3-pyridinylethynyl)phenyl]-1,4-dihydro[1,8]naphthyridin-4-one-3-carboxylate was obtained as a solid.

Step 3: 1-[3-(1-Oxido-3-pyridinylethynyl)phenyl]-1,4-dihydro[1,8]naphthyridin-4-one-3-carboxylic acid.

Following the procedure of Step 3 of EXAMPLE 1C, but substituting the ethyl 1-[3-(1-oxido-3-pyridinylethynyl)phenyl]-1,4-dihydro[1,8]naphthyridin-4-one-3-carboxylate ester from present Step 2 for ethyl 1-(3-bromophenyl)-1,4-dihydro[1,8]naphthyridin-4-one-3-carboxylate, the 1-[3-(1-oxido-3-pyridinylethynyl)phenyl]-1,4-dihydro[1,8]naphthyridin-4-one-3-carboxylic acid was obtained as a white solid. 1H NMR (DMSO-d₆) δ 7.46 (t, 1H), 7.51 (d, 1H), 7.70 (t, 1H), 7.75 (m, 2H), 7.80 (d, 1H), 7.92 (s, 1H), 8.26 (d, 1H), 8.47 (s, 1H), 8.81 (dd, 1H), 8.89 (m, 1H), 8.97 (s, 1H).

1. A method for the treatment of psychiatric disorder in a patient, said method comprises administering to said patient a therapeutically effective amount of a PDE4 inhibitor in combination with psychotherapy.

2. The method of claim 1 wherein said psychiatric disorder is posttraumatic stress disorder.

3. The method of claim 1 wherein said psychotherapy is selected from behavior psychotherapy, cognitive psychotherapy, and psychodynamically oriented psychotherapy.

4. The method of claim 2 wherein said psychotherapy is selected from behavior psychotherapy, cognitive psychotherapy, and psychodynamically oriented psychotherapy.

5. The method of claim 1 wherein said psychotherapy is exposure-based psychotherapy.

6. The method of claim 2 wherein said psychotherapy is exposure-based psychotherapy.

7. The method of claim 1 wherein said psychotherapy is cognitive psychotherapy.

8. The method of claim 2 wherein said psychotherapy is cognitive psychotherapy.

9. The method of claim 1 wherein said PDE4 inhibitor is selected from cilomilast, roflumilast and roflumilast N-oxide.

10. The method of claim 1 wherein said PDE4 inhibitor is selected from cilomilast, roflumilast and roflumilast N-oxide.

11. The method of claim 1 wherein said PDE4 inhibitor is selected from the group consisting of:
6-isopropyl-8-(3-{(Z/E)-2-[4-(methylsulfonyl)phenyl]-2-phenylethenyl}phenyl)quinoline;
6-isopropyl-8-{3-[(E/Z)-2-[4-(methylsulfonyl)phenyl]-2-(1,3-thiazol-2-yl)ethenyl]-phenyl}quinoline;
6-isopropyl-8-(3-{(E)-2-(1-methyl-1H-imidazol-2-yl)-2-[4-(methylsulfonyl)phenyl]ethenyl}-phenyl)quinoline;
6-isopropyl-8-(3-{(Z/E)-2-(4-fluorophenyl)-2-[4-(methylsulfonyl)phenyl]ethenyl}-phenyl)quinoline;
2-(2-{(E/Z)-2-[3-(6-isopropyl-8-quinolinyl)phenyl]-1-[4-(methylsulfonyl)phenyl]ethenyl}-1,3-thiazol-5-yl)-2-propanol;
2-[8-(3-{(E/Z)-2-[5-(1-hydroxy-1-methylethyl)-1,3-thiazol-2-yl]-2-[4-(methylsulfonyl)phenyl]ethenyl}phenyl)-6-quinolinyl]-2-methylpropanenitrile;
2-methyl-2-[8-(3-{(E)-2-(1-methyl-1H-imidazol-2-yl)-2-[4-(methylsulfonyl)phenyl]ethenyl}phenyl)-6-quinolinyl]propanenitrile;
6-[1-(methylsulfonyl)ethyl]-8-{3-[(E)-2-[4-(methylsulfonyl)phenyl]-2-(1,3-thiazol-2-yl)ethenyl]phenyl}quinoline;
6-[1-methyl-1-(methylsulfonyl)ethyl]-8-{3-[(E)-2-[4-(methylsulfonyl)phenyl]-2-(1,3-thiazol-2-yl)ethenyl]phenyl}quinoline;
8-(3-{(Z)-2-(1-methyl-1H-imidazol-2-yl)-2-[4-(methylsulfonyl)phenyl]ethenyl}phenyl)-6-[1-(methylsulfonyl)ethyl]quinoline;
8-(3-{(Z)-2-(1-methyl-1H-imidazol-2-yl)-2-[4-(methylsulfonyl)phenyl]ethenyl}phenyl)-6-[1-methyl-1-(methylsulfonyl)ethyl]quinoline;
6-[1-methyl-1-(methylsulfonyl)ethyl]-8-(3-{(E/Z)-2-(3-methyl-1,2,4-oxadiazol-5-yl)-2-[4-(methylsulfonyl)phenyl]ethenyl}phenyl)quinoline;
(E/Z)-3-{3-[6-(1-cyano-1-methylethyl)-8-quinolinyl]phenyl}-N-isopropyl-2-[4-(methylsulfonyl)phenyl]-2-propenamide;
8-(3-{(E)-2-{3-[(4-methoxyphenoxy)methyl]-1,2,4-oxadiazol-5-yl}-2-[4-(methylsulfonyl)phenyl]ethenyl}phenyl)-6-[1-methyl-1-(methylsulfonyl)ethyl]quinoline;
(5-{(E)-2-(3-{6-[1-methyl-1-(methylsulfonyl)ethyl]-8-quinolinyl}phenyl)-1-[4-(methylsulfonyl)phenyl]ethenyl}-1,2,4-oxadiazol-3-yl)methanol;
(E)-N-isopropyl-3-(3-{6-[1-methyl-1-(methylsulfonyl)ethyl]-8-quinolinyl}phenyl)-2-[4-(methylsulfonyl)phenyl]-2-propenamide;
(E)-3-{3-[6-(1-cyano-1-methylethyl)-8-quinolinyl]phenyl}-2-[4-(methylsulfonyl)phenyl]-2-propenoic acid;
2-methyl-2-[8-(3-{(E)-2-(3-methyl-1,2,4-oxadiazol-5-yl)-2-[4-(methylsulfonyl)phenyl]ethenyl}phenyl)-6-quinolinyl]propanenitrile;
(E)-3-{3-[6-(1-cyano-1-methylethyl)-8-quinolinyl]phenyl}-2-[4-(methylsulfonyl)phenyl]-2-propenamide;
E)-N-(tert-butyl)-3-{3-[6-(1-cyano-1-methylethyl)-8-quinolinyl]phenyl}-2-[4-(methylsulfonyl)phenyl]-2-propenamide;
(E)-3-[3-(6-isopropyl-8-quinolinyl)phenyl]-2-[4-(methylsulfonyl)phenyl]-2-propenoic acid;
6-isopropyl-8-(3-{(E)-2-(3-methyl-1,2,4-oxadiazol-5-yl)-2-[4-(methylsulfonyl)phenyl]ethenyl}phenyl)quinoline;
(E)-3-(3-{6-[1-methyl-1-(methylsulfonyl)ethyl]-8-quinolinyl}phenyl)-2-[4-(methylsulfonyl)phenyl]-1-(1-pyrrolidinyl)-2-propen-1-one;
(E)-N-cyclopropyl-3-(3-{6-[8-methyl-1-(methylsulfonyl)ethyl]-8-quinolinyl}phenyl)-2-[4-(methylsulfonyl)phenyl]-2-propenamide;
(E)-N-(tert-butyl)-3-(3-{6-[1-methyl-1-(methylsulfonyl)ethyl]-8-quinolinyl}phenyl)-2-[4-(methylsulfonyl)phenyl]-2-propenamide;
8-{3-[2,2-bis(4-chlorophenyl)vinyl]phenyl}-6-isopropylquinoline;
6-isopropyl-8-(3-{(E/Z)-2-(6-methyl-3-pyridinyl)-2-[4-(methylsulfonyl)phenyl]ethenyl}-phenyl)quinoline;
6-isopropyl-8-(3-{(E/Z)-2-(5-methyl-2-pyridinyl)-2-[4-(methylsulfonyl)phenyl]-ethenyl}phenyl)quinoline;
8-(3-{2,2-bis[4-(methylsulfonyl)phenyl]vinyl}phenyl)-6-isopropylquinoline;
2-methyl-2-[8-(3-{(E/Z)-2-(5-methyl-2-pyridinyl)-2-[4-(methylsulfonyl)phenyl]ethenyl}phenyl)-6-quinolinyl]propanenitrile;
2-[8-(3-{2,2-bis[4-(methylsulfonyl)phenyl]vinyl}phenyl)-6-quinolinyl]-2-methylpropanenitrile;
2-methyl-2-(8-{3-[(E)-2-[4-(methylsulfonyl)phenyl]-2-(2-pyridinyl)ethenyl]phenyl}-6-quinolinyl)propanenitrile;
6-[1-methyl-1-(methylsulfonyl)ethyl]-8-(3-{(E/Z)-2-(5-methyl-2-pyridinyl)-2-[4-(methylsulfonyl)phenyl]ethenyl}phenyl)quinoline;
2-(6-{(E)-2-(3-{6-[1-methyl-1-(methylsulfonyl)ethyl]-8-quinolinyl}phenyl)-1-[4-(methylsulfonyl)phenyl]ethenyl}-3-pyridinyl)-2-propanol;
(±)-4-{2-[3,4-Bis(difluoromethoxy)phenyl]-2-{5-[2-(1-hydroxy-1-methyl)ethyl]thiazolyl}ethyl}pyridine N-oxide;
chiral 4-{2-[3,4-Bis(difluoromethoxy)phenyl]-2-{5-[2-(1-hydroxy-1-methyl)ethyl]thiazolyl}ethyl}pyridine N-oxide;
(±/±)-4-{2-[3,4-Bis(difluoromethoxy)phenyl]-2-[5-(2-(1-hydroxy-2,2,2-trifluoro)ethyl)thiazolyl]ethyl}pyridine;
(±/±)-4-{2-[3,4-Bis(difluoromethoxy)phenyl]-2-[5-(2-(1-hydroxy-2,2,2-trifluoro)-ethyl)thiazolyl]ethyl}pyridine N-oxide;
(±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine;
(±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine N-oxide;
(±/±)-4-{2-[3,4-Bis(difluoromethoxy)phenyl]-2-{5-[2-(1-hydroxy-1-trifluoromethyl)ethyl]-thiazolyl}ethyl}pyridine N-oxide;
(±/±)-4-{2-[3,4-Bis(difluoromethoxy)phenyl]-2-[5-(2-phenylmethanol)thiazolyl]ethyl}pyridine N-oxide;
(±/±)-4-{2-[3,4-Bis(difluoromethoxy)phenyl]-2-[5-(2-(1-hydroxy-1-phenyl)ethyl)-thiazolyl]ethyl}pyridine N-oxide;
(±/±)-4-{2-[3,4-Bis(difluoromethoxy)phenyl]-2-[5-(2-(1-hydroxy-1-phenyl-2,2,2-trifluoro)ethyl)thiazolyl]ethyl}pyridine N-oxide;
(±/±)-4-{2-[3,4-Bis(difluoromethoxy)phenyl]-2-[5-(2-(1-hydroxy-1-phenyl)propyl)thiazolyl]ethyl}pyridine N-oxide;
(±/±)-4-{2-[3,4-Bis(difluoromethoxy)phenyl]-2-[5-(2-cyclohexylmethanol)thiazolyl]ethyl}pyridine;
(±/±)-4-{2-[3,4-Bis(difluoromethoxy)phenyl]-2-[5-(2-(1-hydroxy-1-cyclohexyl-2,2,2-trifluoromethyl)ethyl)thiazolyl]ethyl}pyridine N-oxide;
(±/±)-4-{2-[3,4-Bis(difluoromethoxy)phenyl]-2-[5-(2-(1-hydroxy-1-(4-ethyl)phenyl)ethyl)thiazolyl]ethyl}pyridine N-oxide;
(±/±)-4-{2-[3,4-Bis(difluoromethoxy)phenyl]-2-[5-(2-(1-hydroxy-1-(4-ethyl)phenyl-2,2,2-trifluoro)ethyl)thiazolyl]ethyl}pyridine N-oxide;
(±/±)-4-{2-[3,4-Bis(difluoromethoxy)phenyl]-2-[5-(2-(1-hydroxy-1-(4-fluoro)phenyl)-ethyl)thiazolyl]ethyl}pyridine N-oxide;
(±/±)-4-{2-[3,4-Bis(difluoromethoxy)phenyl]-2-[5-(2-(1-hydroxy-1-(4-fluoro)phenyl-2,2,2-trifluoro)ethyl)thiazolyl]ethyl}pyridine N-oxide;
(±/±)-4-{2-[3,4-Bis(difluoromethoxy)phenyl]-2-[5-(2-(1-hydroxy-1-(5-bromopyridin-2-yl)-2,2,2-trifluoro)ethyl)thiazolyl]ethyl}pyridine N-oxide;
(±/±)-4-{2-[3,4-Bis(difluoromethoxy)phenyl]-2-[5-(2-(1-hydroxy-1-(6-bromopyridin-3-yl)-2,2,2-trifluoro)ethyl)thiazolyl]ethyl}pyridine N-oxide;
(±)-4-{2-[3,4-Bis(difluoromethoxy)phenyl]-2-{5-[2-(1-hydroxy)cyclobutyl]thiazolyl}ethyl}pyridine N-oxide;
(±)-4-{2-[3,4-Bis(difluoromethoxy)phenyl]-2-{5-[2-(1-hydroxy)cyclohexyl]thiazolyl}ethyl}pyridine N-oxide;
(±)-4-{2-[(3-cyclobutyloxy-4-difluoromethoxy)phenyl]-2-{5-[2-(1-hydroxy-1-methyl)ethyl]thiazolyl}ethyl}pyridine N-oxide;
chiral 4-{2-[(3-cyclobutyloxy-4-difluoromethoxy)phenyl]-2-{5-[2-(1-hydroxy-1-methyl)ethyl]thiazolyl}ethyl}pyridine N-oxide;
(±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine;
(±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
chiral 3-{2-[(3-cyclobutyloxy-4-difluoromethoxy)phenyl]-2-{5-[2-(1-hydroxy-1-methyl)ethyl]thiazolyl}ethyl}pyridine;
chiral 3-{2-[(3-cyclobutyloxy-4-difluoromethoxy)phenyl]-2-{5-[2-(1-hydroxy-1-methyl)ethyl]thiazolyl}ethyl}pyridine N-oxide;
chiral 3-{2-[(3-cyclobutyloxy-4-difluoromethoxy)phenyl]-2-{5-[2-(1-hydroxy-1-methyl)ethyl]thiazolyl}ethyl}pyridine N-oxide;
chiral 3-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
(±)-4-{2-[(3-cyclopropyloxy-4-difluoromethoxy)phenyl]-2-{5-[2-(1-hydroxy-1-methyl)ethyl]thiazolyl}ethyl}pyridine N-oxide;
(±)-3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
chiral 3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
N-Isopropyl-1-[3-(phenylethynyl)phenyl]-1,4-dihydro[1,8]naphthyridin-4-one-3-carboxamide;
N-Isopropyl-1-[3-(2-pyridinylethynyl)phenyl]-1,4-dihydro[1,8]naphthyridin-4-one-3-carboxamide;
N-Isopropyl-1-[3-(4-pyridinylethynyl)phenyl]-1,4-dihydro[1,8]naphthyridin-4-one-3-carboxamide;
N-Isopropyl-1-[3-(1-oxido-4-pyridinylethynyl)phenyl]-1,4-dihydro[1,8]naphthyridin-4-one-3-carboxamide;
N-Isopropyl-1-(3-ethynylphenyl)-1,4-dihydro[1,8]naphthyridin-4-one-3-carboxamide;
N-Cyclopropyl-1-(3-ethynylphenyl)-1,4-dihydro[1,8]naphthyridin-4-one-3-carboxamide;
N-Isopropyl-1-[3-(3-pyridinylethynyl)phenyl]-1,4-dihydro[1,8]naphthyridin-4-one-3-carboxamide;
N-Isopropyl-1-[3-(1-oxido-3-pyridinylethynyl)phenyl]-1,4-dihydro[1,8]naphthyridin-4-one-3-carboxamide;
N-Cyclopropyl-1-[3-(3-pyridinylethynyl)phenyl]-1,4-dihydro[1,8]naphthyridin-4-one-3-carboxamide;
N-Isopropyl-1-[3-(3-hydroxy-3-methylbut-1-ynyl)phenyl]-1,4-dihydro[1,8]naphthyridin-4-one-3-carboxamide;
N-Cyclopropyl-1-[3-(3-hydroxy-3-methylbut-1-ynyl)phenyl]-1,4-dihydro[1,8]naphthyridin-4-one-3-carboxamide;
N-Isopropyl-1-[3-(1-hydroxycyclopentyl)ethynylphenyl]-1,4-dihydro[1,8]naphthyridin-4-one-3-carboxamide;
N-Isopropyl-1-[3-(1-hydroxycyclopropyl)ethynylphenyl]-1,4-dihydro[1,8]naphthyridin-4-one-3-carboxamide;
N-Isopropyl-1-{3-[4,4,4-trifluoro-3-hydroxy-3-(trifluoromethyl)but-1-ynyl]phenyl}-1,4-dihydro[1,8]naphthyridin-4-one-3-carboxamide;
N-Isopropyl-1-[3-(3-hydroxy-3-phenylbut-1-ynyl)phenyl]-1,4-dihydro[1,8]naphthyridin-4-one-3-carboxamide;
N-Cyclopropyl-1-[3-(1-oxido-3-pyridinylethynyl)phenyl]-1,4-dihydro[1,8]naphthyridin-4-one-3-carboxamide;
N-Isopropyl-1-[3-(3-amino-3-ethylpent-1-ynyl)phenyl]-1,4-dihydro[1,8]naphthyridin-4-one-3-carboxamide; or
N-Cyclopropyl-1-[3-(3-amino-3-ethylpent-1-ynyl)phenyl]-1,4-dihydro[1,8]naphthyridin-4-one-3-carboxamide;
N-Isopropyl-1-[3-(3-quinolinylethynyl)phenyl]-1,4-dihydro[1,8]naphthyridin-4-one-3-carboxamide; or
N-Isopropyl-1-[3-(1-oxido-3-quinolinylethynyl)phenyl]-1,4-dihydro[1,8]naphthyridin-4-one-3-carboxamide;
N-Isopropyl-1-[3-(cyclopropylethynyl)phenyl]-1,4-dihydro[1,8]naphthyridin-4-one-3-carboxamide;
N-Isopropyl-1-[3-(6-amino-3-pyridinylethynyl)phenyl]-1,4-dihydro[1,8]naphthyridin-4-one-3-carboxamide;
N-Isopropyl-1-{3-[5-(1-hydroxy-1-methylethyl)-1-oxido-3-pyridinylethynyl]phenyl}-1,4-dihydro[1,8]naphthyridin-4-one-3-carboxamide;
N-Isopropyl-1-{3-[6-(1-hydroxy-1-methylethyl)-3-pyridinylethynyl]phenyl}-1,4-dihydro[1,8]-naphthyridin-4-one-3-carboxamide;
N-Isopropyl-1-{3-[6-(1-hydroxy-1-methylethyl)-1-oxido-3-pyridinylethynyl]phenyl}-1,4-dihydro[1,8]naphthyridin-4-one-3-carboxamide;
N-Isopropyl-1-{3-[4-(1-hydroxy-1-methylethyl)-2-pyridinylethynyl]phenyl}-1,4-dihydro[1,8]-naphthyridin-4-one-3-carboxamide;
N-Isopropyl-1-{3-[5-(1-hydroxy-1-methylethyl)-2-pyridinylethynyl]phenyl}-1,4-dihydro[1,8]-naphthyridin-4-one-3-carboxamide;
N-Isopropyl-1-{3-[6-(1-hydroxy-1-methylethyl)-2-pyridinylethynyl]phenyl}-1,4-dihydro[1,8]-naphthyridin-4-one-3-carboxamide;
N-Cyclopropyl-1-{3-[6-(1-hydroxy-1-methylethyl)-1-oxido-3-pyridinylethynyl]phenyl}-1,4-dihydro[1,8]naphthyridin-4-one-3-carboxamide;
N-Isopropyl-1-{3-[(4-pyridin-3-ylphenyl)ethynyl]phenyl}-1,4-dihydro[1,8]naphthyridin-4-one-3-carboxamide;
N-Isopropyl-1-(3-{[5-(1-hydroxy-1-methylethyl)thien-2-yl]ethynyl}phenyl)-1,4-dihydro[1,8]-naphthyridin-4-one-3-carboxamide;
N-Isopropyl-1-(3-{[2-(1-hydroxy-1-methylethyl)-1,3-thiazol-5-yl]ethynyl}phenyl)-1,4-dihydro[1,8]naphthyridin-4-one-3-carboxamide;
1-[3-(1-Oxido-3-pyridinylethynyl)phenyl]-1,4-dihydro[1,8]naphthyridin-4-one-3-carboxamide; and
1-[3-(1-Oxido-3-pyridinylethynyl)phenyl]-1,4-dihydro[1,8]naphthyridin-4-one-3-carboxylic acid,
or a pharmaceutically acceptable salt thereof.

12. The method of claim 1 wherein said PDE4 inhibitor is selected from the group consisting of:
6-isopropyl-8-(3-{(Z/E)-2-[4-(methylsulfonyl)phenyl]-2-phenylethenyl}phenyl)quinoline;
6-isopropyl-8-{3-[(E/Z)-2-[4-(methylsulfonyl)phenyl]-2-(1,3-thiazol-2-yl)ethenyl]-phenyl}quinoline;
6-isopropyl-8-(3-{(E)-2-(1-methyl-1H-imidazol-2-yl)-2-[4-(methylsulfonyl)phenyl]ethenyl}-phenyl)quinoline;
6-isopropyl-8-(3-{(Z/E)-2-(4-fluorophenyl)-2-[4-(methylsulfonyl)phenyl]ethenyl}-phenyl)quinoline;
2-(2-{(E/Z)-2-[3-(6-isopropyl-8-quinolinyl)phenyl]-1-[4-(methylsulfonyl)phenyl]ethenyl}-1,3-thiazol-5-yl)-2-propanol;
2-[8-(3-{(E/Z)-2-[5-(1-hydroxy-1-methylethyl)-1,3-thiazol-2-yl]-2-[4-(methylsulfonyl)phenyl]ethenyl}phenyl)-6-quinolinyl]-2-methylpropanenitrile;
2-methyl-2-[8-(3-{(E)-2-(1-methyl-1H-imidazol-2-yl)-2-[4-(methylsulfonyl)phenyl]ethenyl}phenyl)-6-quinolinyl]propanenitrile;
6-[1-(methylsulfonyl)ethyl]-8-{3-[(E)-2-[4-(methylsulfonyl)phenyl]-2-(1,3-thiazol-2-yl)-ethenyl]phenyl}quinoline;
6-[1-methyl-1-(methylsulfonyl)ethyl]-8-{3-[(E)-2-[4-(methylsulfonyl)phenyl]-2-(1,3-thiazol-2-yl)ethenyl]phenyl}quinoline;
8-(3-{(Z)-2-(1-methyl-1H-imidazol-2-yl)-2-[4-(methylsulfonyl)phenyl]ethenyl}phenyl)-6-[1-(methylsulfonyl)ethyl]quinoline;
8-(3-{(Z)-2-(1-methyl-1H-imidazol-2-yl)-2-[4-(methylsulfonyl)phenyl]ethenyl}phenyl)-6-[1-methyl-1-(methylsulfonyl)ethyl]quinoline;
6-[1-methyl-1-(methylsulfonyl)ethyl]-8-(3-{(E/Z)-2-(3-methyl-1,2,4-oxadiazol-5-yl)-2-[4-(methylsulfonyl)phenyl]ethenyl}phenyl)quinoline;
(E/Z)-3-{3-[6-(1-cyano-1-methylethyl)-8-quinolinyl]phenyl}-N-isopropyl-2-[4-(methylsulfonyl)phenyl]-2-propenamide;
8-(3-{(E)-2-{3-[(4-methoxyphenoxy)methyl]-1,2,4-oxadiazol-5-yl}-2-[4-(methylsulfonyl)phenyl]ethenyl}phenyl)-6-[1-methyl-1-(methylsulfonyl)ethyl]quinoline;
(5-{(E)-2-(3-{6-[1-methyl-1-(methylsulfonyl)ethyl]-8-quinolinyl}phenyl)-1-[4-(methylsulfonyl)phenyl]ethenyl}-1,2,4-oxadiazol-3-yl)methanol;
(E)-N-isopropyl-3-(3-{6-[1-methyl-1-(methylsulfonyl)ethyl]-8-quinolinyl}phenyl)-2-[4-(methylsulfonyl)phenyl]-2-propenamide;
(E)-3-{3-[6-(1-cyano-1-methylethyl)-8-quinolinyl]phenyl}-2-[4-(methylsulfonyl)phenyl]-2-propenoic acid;
2-methyl-2-[8-(3-{(E)-2-(3-methyl-1,2,4-oxadiazol-5-yl)-2-[4-(methylsulfonyl)phenyl]ethenyl}phenyl)-6-quinolinyl]propanenitrile;
(E)-3-{3-[6-(1-cyano-1-methylethyl)-8-quinolinyl]phenyl}-2-[4-(methylsulfonyl)phenyl]-2-propenamide;
E)-N-(tert-butyl)-3-{3-[6-(1-cyano-1-methylethyl)-8-quinolinyl]phenyl}-2-[4-(methylsulfonyl)phenyl]-2-propenamide;
(E)-3-[3-(6-isopropyl-8-quinolinyl)phenyl]-2-[4-(methylsulfonyl)phenyl]-2-propenoic acid;
6-isopropyl-8-(3-{(E)-2-(3-methyl-1,2,4-oxadiazol-5-yl)-2-[4-(methylsulfonyl)phenyl]ethenyl}phenyl)quinoline;
(E)-3-(3-{6-[1-methyl-1-(methylsulfonyl)ethyl]-8-quinolinyl}phenyl)-2-[4-(methylsulfonyl)phenyl]-1-(1-pyrrolidinyl)-2-propen-1-one;
(E)-N-cyclopropyl-3-(3-{6-[1-methyl-1-(methylsulfonyl)ethyl]-8-quinolinyl}phenyl)-2-[4-(methylsulfonyl)phenyl]-2-propenamide;
(E)-N-(tert-butyl)-3-(3-{6-[1-methyl-1-(methylsulfonyl)ethyl]-8-quinolinyl}phenyl)-2-[4-(methylsulfonyl)phenyl]-2-propenamide;
8-{3-[2,2-bis(4-chlorophenyl)vinyl]phenyl}-6-isopropylquinoline;
6-isopropyl-8-(3-{(E/Z)-2-(6-methyl-3-pyridinyl)-2-[4-(methylsulfonyl)phenyl]ethenyl}phenyl)quinoline;
6-isopropyl-8-(3-{(E/Z)-2-(5-methyl-2-pyridinyl)-2-[4-(methylsulfonyl)phenyl]ethenyl}phenyl)quinoline;
8-(3-{2,2-bis[4-(methylsulfonyl)phenyl]vinyl}phenyl)-6-isopropylquinoline;
2-methyl-2-[8-(3-{(E/Z)-2-(5-methyl-2-pyridinyl)-2-[4-(methylsulfonyl)phenyl]ethenyl}phenyl)-6-quinolinyl]propanenitrile;
2-[8-(3-{2,2-bis[4-(methylsulfonyl)phenyl]vinyl}phenyl)-6-quinolinyl]-2-methylpropanenitrile;
2-methyl-2-(8-{3-[(E)-2-[4-(methylsulfonyl)phenyl]-2-(2-pyridinyl)ethenyl]phenyl}-6-quinolinyl)propanenitrile;
6-[1-methyl-1-(methylsulfonyl)ethyl]-8-(3-{(E/Z)-2-(5-methyl-2-pyridinyl)-2-[4-(methylsulfonyl)phenyl]ethenyl}phenyl)quinoline;
2-(6-{(E)-2-(3-{6-[1-methyl-1-(methylsulfonyl)ethyl]-8-quinolinyl}phenyl)-1-[4-(methylsulfonyl)phenyl]ethenyl}-3-pyridinyl)-2-propanol;
(±)-4-{2-[3,4-Bis(difluoromethoxy)phenyl]-2-{5-[2-(1-hydroxy-1-methyl)ethyl]thiazolyl}ethyl}pyridine N-oxide;
chiral 4-{2-[3,4-Bis(difluoromethoxy)phenyl]-2-{5-[2-(1-hydroxy-1-methyl)ethyl]thiazoly}ethyl}pyridine N-oxide;
(±/±)-4-{2-[3,4-Bis(difluoromethoxy)phenyl]-2-[5-(2-(1-hydroxy-2,2,2-trifluoro)ethyl)thiazolyl]ethyl}pyridine;
(±/±)-4-{2-[3,4-Bis(difluoromethoxy)phenyl]-2-[5-(2-(1-hydroxy-2,2,2-trifluoro)ethyl)thiazolyl]ethyl}pyridine N-oxide;
(±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine;
(±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine N-oxide;
(±/±)-4-{2-[3,4-Bis(difluoromethoxy)phenyl]-2-{5-[2-(1-hydroxy-1-trifluoromethyl)ethyl]thiazolyl}ethyl}pyridine N-oxide;
(±/±)-4-{2-[3,4-Bis(difluoromethoxy)phenyl]-2-[5-(2-phenylmethanol)thiazolyl]ethyl}pyridine N-oxide;
(±/±)-4-{2-[3,4-Bis(difluoromethoxy)phenyl]-2-[5-(2-(1-hydroxy-1-phenyl)ethyl)thiazolyl]ethyl}pyridine N-oxide;
(±/±)-4-{2-[3,4-Bis(difluoromethoxy)phenyl]-2-[5-(2-(1-hydroxy-1-phenyl-2,2,2-trifluoro)ethyl)thiazolyl]ethyl}pyridine N-oxide;
(±/±)-4-{2-[3,4-Bis(difluoromethoxy)phenyl]-2-[5-(2-(1-hydroxy-1-phenyl)propyl)thiazolyl]ethyl}pyridine N-oxide;
(±/±)-4-{2-[3,4-Bis(difluoromethoxy)phenyl]-2-[5-(2-cyclohexylmethanol)thiazolyl]ethyl}pyridine;
(±/±)-4-{2-[3,4-Bis(difluoromethoxy)phenyl]-2-[5-(2-(1-hydroxy-1-cyclohexyl-2,2,2-trifluoromethyl)ethyl)thiazolyl]ethyl}pyridine N-oxide;
(±/±)-4-{2-[3,4-Bis(difluoromethoxy)phenyl]-2-[5-(2-(1-hydroxy-1-(4-ethyl)phenyl)ethyl)thiazolyl]ethyl}pyridine N-oxide;
(±/±)-4-{2-[3,4-Bis(difluoromethoxy)phenyl]-2-[5-(2-(1-hydroxy-1-(4-ethyl)phenyl-2,2,2-trifluoro)ethyl)thiazolyl]ethyl}pyridine N-oxide;
(±/±)-4-{2-[3,4-Bis(difluoromethoxy)phenyl]-2-[5-(2-(1-hydroxy-1-(4-fluoro)phenyl)ethyl)thiazolyl]ethyl}pyridine N-oxide;
(±/±)-4-{2-[3,4-Bis(difluoromethoxy)phenyl]-2-[5-(2-(1-hydroxy-1-(4-fluoro)phenyl-2,2,2-trifluoro)ethyl)thiazolyl]ethyl}pyridine N-oxide;
(±/±)-4-{2-[3,4-Bis(difluoromethoxy)phenyl]-2-[5-(2-(1-hydroxy-1-(5-bromopyridin-2-yl)-2,2,2-trifluoro)ethyl)thiazolyl]ethyl}pyridine N-oxide;
(±/±)-4-{2-[3,4-Bis(difluoromethoxy)phenyl]-2-[5-(2-(1-hydroxy-1-(6-bromopyridin-3-yl)-2,2,2-trifluoro)ethyl)thiazolyl]ethyl}pyridine N-oxide;
(±)-4-{2-[3,4-Bis(difluoromethoxy)phenyl]-2-{5-[2-(1-hydroxy)cyclobutyl]thiazolyl}-ethyl}pyridine N-oxide;
(±)-4-{2-[3,4-Bis(difluoromethoxy)phenyl]-2-{5-[2-(1-hydroxy)cyclohexyl]thiazolyl}ethyl)}pyridine N-oxide;
(±)-4-{2-[(3-cyclobutyloxy-4-difluoromethoxy)phenyl]-2-{5-[2-(1-hydroxy-1-methyl)ethyl]thiazolyl}ethyl}pyridine N-oxide;
chiral 4-{2-[(3-cyclobutyloxy-4-difluoromethoxy)phenyl]-2-{5-[2-(1-hydroxy-1-methyl)ethyl]thiazolyl}ethyl}pyridine N-oxide;
(±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine;
(±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
chiral 3-{2-[(3-cyclobutyloxy-4-difluoromethoxy)phenyl]-2-{5-[2-(1-hydroxy-1-methyl)ethyl]thiazolyl}ethyl}pyridine;
chiral 3-{2-[(3-cyclobutyloxy-4-difluoromethoxy)phenyl]-2-{5-[2-(1-hydroxy-1-methyl)ethyl]thiazolyl}ethyl}pyridine N-oxide;
chiral 3-{2-[(3-cyclobutyloxy-4-difluoromethoxy)phenyl]-2-{5-[2-(1-hydroxy-1methyl)ethyl]thiazolyl}ethyl}pyridine N-oxide;
chiral 3-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
(±)-4-{2-[(3-cyclopropyloxy-4-difluoromethoxy)phenyl]-2-{5-[2-(1-hydroxy-1-methyl)ethyl]thiazolyl}ethyl}pyridine N-oxide;
(±)-3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
chiral 3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
N-Isopropyl-1-[3-(phenylethynyl)phenyl]-1,4-dihydro[1,8]naphthyridin-4-one-3-carboxamide;
N-Isopropyl-1-[3-(2-pyridinylethynyl)phenyl]-1,4-dihydro[1,8]naphthyridin-4-one-3-carboxamide;
N-Isopropyl-1-[3-(4-pyridinylethynyl)phenyl]-1,4-dihydro[1,8]naphthyridin-4-one-3-carboxamide;
N-Isopropyl-1-[3-(1-oxido-4-pyridinylethynyl)phenyl]-1,4-dihydro[1,8]naphthyridin-4-one-3-carboxamide;
N-Isopropyl-1-(3-ethynylphenyl)-1,4-dihydro[1,8]naphthyridin-4-one-3-carboxamide;
N-Cyclopropyl-1-(3-ethynylphenyl)-1,4-dihydro[1,8]naphthyridin-4-one-3-carboxamide;
N-Isopropyl-1-[3-(3-pyridinylethynyl)phenyl]-1,4-dihydro[1,8]naphthyridin-4-one-3-carboxamide;
N-Isopropyl-1-[3-(1-oxido-3-pyridinylethynyl)phenyl]-1,4-dihydro[1,8]naphthyridin-4-one-3-carboxamide;
N-Cyclopropyl-1-[3-(3-pyridinylethynyl)phenyl]-1,4-dihydro[1,8]naphthyridin-4-one-3-carboxamide;
N-Isopropyl-1-[3-(3-hydroxy-3-methylbut-1-ynyl)phenyl]-1,4-dihydro[1,8]naphthyridin-4-one-3-carboxamide;
N-Cyclopropyl-1-[3-(3-hydroxy-3-methylbut-1-ynyl)phenyl]-1,4-dihydro[1,8]naphthyridin-4-one-3-carboxamide;
N-Isopropyl-1-[3-(1-hydroxycyclopentyl)ethynylphenyl]-1,4-dihydro[1,8]naphthyridin-4-one-3-carboxamide;
N-Isopropyl-1-[3-(1-hydroxycyclopropyl)ethynylphenyl]-1,4-dihydro[1,8]naphthyridin-4-one-3-carboxamide;
N-Isopropyl-1-{3-[4,4,4-trifluoro-3-hydroxy-3-(trifluoromethyl)but-1-ynyl]phenyl}-1,4-dihydro[1,8]naphthyridin-4-one-3-carboxamide;
N-Isopropyl-1-[3-(3-hydroxy-3-phenylbut-1-ynyl)phenyl]-1,4-dihydro[1,8]naphthyridin-4-one-3-carboxamide;
N-Cyclopropyl-1-[3-(1-oxido-3-pyridinylethynyl)phenyl]-1,4-dihydro[1,8]naphthyridin-4-one-3-carboxamide;
N-Isopropyl-1-[3-(3-amino-3-ethylpent-1-ynyl)phenyl]-1,4-dihydro[1,8]naphthyridin-4-one-3-carboxamide; or
N-Cyclopropyl-1-[3-(3-amino-3-ethylpent-1-ynyl)phenyl]-1,4-dihydro[1,8]naphthyridin-4-one-3-carboxamide;
N-Isopropyl-1-[3-(3-quinolinylethynyl)phenyl]-1,4-dihydro[1,8]naphthyridin-4-one-3-carboxamide; or
N-Isopropyl-1-[3-(1-oxido-3-quinolinylethynyl)phenyl]-1,4-dihydro[1,8]naphthyridin-4-one-3-carboxamide;
N-Isopropyl-1-[3-(cyclopropylethynyl)phenyl]-1,4-dihydro[1,8]naphthyridin-4-one-3-carboxamide;
N-Isopropyl-1-[3-(6-amino-3-pyridinylethynyl)phenyl]-1,4-dihydro[1,8]naphthyridin-4-one-3-carboxamide;
N-Isopropyl-1-{3-[5-(1-hydroxy-1-methylethyl)-1-oxido-3-pyridinylethynyl]phenyl}-1,4-dihydro[1,8]naphthyridin-4-one-3-carboxamide;
N-Isopropyl-1-{3-[6-(1-hydroxy-1-methylethyl)-3-pyridinylethynyl]phenyl}-1,4-dihydro[1,8]-naphthyridin-4-one-3-carboxamide;
N-Isopropyl-1-{3-[6-(1-hydroxy-1-methylethyl)-1-oxido-3-pyridinylethynyl]phenyl}-1,4-dihydro[1,8]naphthyridin-4-one-3-carboxamide;
N-Isopropyl-1-{3-[4-(1-hydroxy-1-methylethyl)-2-pyridinylethynyl]phenyl}-1,4-dihydro[1,8]-naphthyridin-4-one-3-carboxamide;
N-Isopropyl-1-{3-[5-(1-hydroxy-1-methylethyl)-2-pyridinylethynyl]phenyl}-1,4-dihydro[1,8]-naphthyridin-4-one-3-carboxamide;
N-Isopropyl-1-{3-[6-(1-hydroxy-1-methylethyl)-2-pyridinylethynyl]phenyl}-1,4-dihydro[1,8]-naphthyridin-4-one-3-carboxamide;
N-Cyclopropyl-1-{3-[6-(1-hydroxy-1-methylethyl)-1-oxido-3-pyridinylethynyl]phenyl}-1,4-dihydro[1,8]naphthyridin-4-one-3-carboxamide;
N-Isopropyl-1-{3-[(4-pyridin-3-ylphenyl)ethynyl]phenyl}-1,4-dihydro[1,8]naphthyridin-4-one-3-carboxamide;
N-Isopropyl-1-(3-{[5-(1-hydroxy-1-methylethyl)thien-2-yl]ethynyl}phenyl)-1,4-dihydro[1,8]-naphthyridin-4-one-3-carboxamide;
N-Isopropyl-1-(3-{[2-(1-hydroxy-1-methylethyl)-1,3-thiazol-5-yl]ethynyl}phenyl)-1,4-dihydro[1,8]naphthyridin-4-one-3-carboxamide;
1-[3-(1-Oxido-3-pyridinylethynyl)phenyl]-1,4-dihydro[1,8]naphthyridin-4-one-3-carboxamide; and
1-[3-(1-Oxido-3-pyridinylethynyl)phenyl]-1,4-dihydro[1,8]naphthyridin-4-one-3-carboxylic acid,
or a pharmaceutically acceptable salt thereof.
